(19) United States
(12) Patent Application Publication
(10) Pub. No.: US 2008/0318989 A1
(43) Pub. Date: Dec. 25, 2008

(54) Pyrimidine Kinase Inhibitors

(76) Inventors: Daniel J. Burdick, Burlingame, CA (US); Jun Liang, Palo Alto, CA (US)

Correspondence Address: GENENTECH, INC., 1 DNA WAY, SOUTH SAN FRANCISCO, CA, 94080, US

(21) Appl. No.: 12/097,030

(22) PCT Filed: Dec. 15, 2006
(86) PCT No.: PCT/US06/62181

§ 371 Date: Jun. 11, 2008

Related U.S. Application Data
(60) Provisional application No. 60/752,013, filed on Dec. 19, 2005.

(51) Int. Cl. A61K 31/506; C07D 239/24; C12N 5/00; A61P 35/00

(52) U.S. Cl. 514/275; 544/332; 544/323; 435/375

(57) ABSTRACT

The invention provides novel kinase inhibitors that are useful as therapeutic agents for example in the treatment malignancies where the compounds have the general formula (I) wherein ring A, X, Y, Z, R₁, R₂, R₃, R₄, m and n are as defined herein.

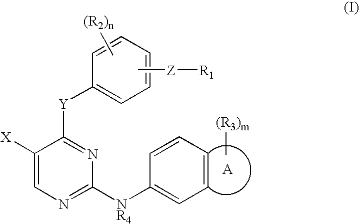

This application claims priority to provisional U.S. patent application No. 60/752,013 filed Dec. 19, 2005, the entirety of which is incorporated herein by reference.

### FIELD OF THE INVENTION

The present invention relates to organic compounds useful for therapy and/or prophylaxis in a mammal, and in particular to inhibitors of kinases useful for treating cancers.

### BACKGROUND OF THE INVENTION

An important class of enzymes that has been the subject of extensive study is protein kinases which are involved in a majority of cellular signaling pathways affecting cell proliferation, migration, differentiation, and metabolism. Kinases function by removing a phosphate group from ATP and phosphorylating hydroxyl groups on serine, threonine and tyrosine amino acid residues of proteins in response to a stimulus such as environmental and chemical stress signals (e.g. osmotic shock, heat shock, ultraviolet radiation, bacterial endotoxin), cytokines (e.g., interleukin-1 and tumor necrosis factor alpha), and growth factors (e.g. granulocyte macrophage-colony-stimulating factor, transforming growth factor, fibroblast growth factor). Many diseases are associated with abnormal cellular responses triggered by protein kinase-mediated events. These diseases include autoimmune diseases, inflammatory diseases, bone diseases, metabolic diseases, neurological and neurodegenerative diseases, cancer, cardiovascular diseases, allergies and asthma, Alzheimer's disease and hormone-related diseases. Accordingly, there has been a substantial effort in medicinal chemistry to find inhibitors of protein kinase that are effective as therapeutic agents.

Aurora kinase is a family serine/threonine kinases that are essential for cell proliferation. The three known mammalian family members, Aurora-A (also referred to as Aurora-2, Aur-2, STK-15), Aurora-B (also referred to as Aurora-1, Aur-1 and STK-12) and Aurora-C (also referred to as STK-13), are highly homologous proteins responsible for chromosome segregation, mitotic spindle function and cytokinesis. (Bischoff, J. R. & Plowman, G. D., Trends in Cell Biology 9:454, 1999; Giet R. and Prigent, C. Journal of Cell Science 112:3591, 1999; Nigg, E. A., Nat. Rev. Mol. Cell. Biol. 2:21, 2001; Adams, R. R. Carmena, M. and Earnshaw, W. C., Trends in Cell Biology 11:49, 2001). Aurora kinase expression is low or undetectable in resting cells, with expression and activity peaking during the G2 and mitotic phases in cycling cells. In mammalian cells, proposed substrates for Aurora kinase include histone H3, a protein involved in chromosome condensation, and CENP-A, myosin II regulatory light chain, I protein phosphatase 1, TPX2, all of which are required for cell division. Aurora-A plays a role in the cell cycle by controlling the accurate segregation of chromosomes during mitosis and misregulation thereof can lead to cellular proliferation and other abnormalities.

Since its discovery in 1997 the mammalian Aurora kinase family has been closely linked to tumorigenesis due to its effect on genetic stability. Cells with elevated levels of this kinase contain multiple centrosomes and multipolar spindles, and rapidly become aneuploid. Indeed, a correlation between amplification of the Aurora-A locus and chromosomal instability in mammary and gastric tumours has been observed. (Miyoshi, Y., Iwao, K., Egawa, C., and Noguchi, S. Int. J. Cancer 92:370, 2001; Sakakura, C. et al. British Journal of Cancer 84:824, 2001). Moreover, Aurora-A overexpression has been shown to transforms rodent fibroblasts (Bischoff, J. R., et al. EMBO J. 17:3052, 1998).

The Aurora kinases have been reported to be overexpressed in a wide range of human tumours. Elevated expression of Aurora-A has been detected in over 50% of colorectal, ovarian and gastric cancers, and in 94% of invasive duct adenocarcinomas of the breast. Amplification and/or overexpression of Aurora-A have also been reported in renal, cervical, neuroblastoma, melanoma, lymphoma, bladder, pancreatic and prostate tumours and is associated with aggressive clinical behaviour. For example, amplification of the aurora-A locus (20q1 3) correlates with poor prognosis for patients with node-negative breast cancer (Isola, J. J., et al. American Journal of Pathology 147:905, 1995). Aurora-B is highly expressed in multiple human tumour cell lines, including colon, breast, lung, melanoma, kidney, ovary, pancreas, CNS, gastric tract and leukemias (Tatsuka et al 1998 58, 4811-4816; Katayama et al., Gene 244:1). Also, levels of Aurora-B enzyme have been shown to increase as a function of Duke's stage in primary colorectal cancers (Katayama, H. et al. Journal of the National Cancer Institute 91:1160, 1999). Aurora-C, which is normally only found in testis, is also overexpressed in a high percentage of primary colorectal cancers and in a variety of tumour cell lines including cervical adenocarcinoma and breast carcinoma cells (Kimura, M., et al., Journal of Biological Chemistry 274:7334, 1999; Takahashi, T., et al., Jpn. J. Cancer Res. 91:1007-1014, 2000).

Based on the known function of the Aurora kinases, inhibition of their activity will disrupt mitosis leading to cell cycle arrest halting cellular proliferation and therefore will slow tumour growth in a wide range of cancers.

### SUMMARY OF THE INVENTION

In one aspect of the present invention there is provided novel inhibitors of Aurora kinases having the general formula (I)

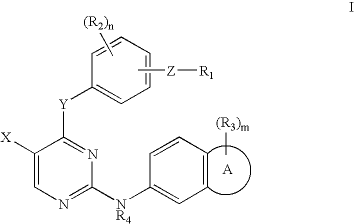

wherein
ring A is a 5, 6 or 7 member ring carbocycle or heterocycle;
X is H, hydroxyl, halo, amino, nitro, alkyl or haloalkyl;
Y is O, S or NR₄;
Z is —NR₄C(O)— or —C(O)NR₄—;
R₁ is alkyl, a carbocycle or a heterocycle optionally substituted with hydroxyl, halogen, oxo, amino, carboxyl or alkoxy;
R₂ is hydroxyl, halogen, amino, carboxyl or is alkyl, acyl, alkoxy or alkylthio optionally substituted with hydroxyl, halogen, oxo, thione, amino, carboxyl or alkoxy;
R₃ is hydroxyl, halogen, amino, oxo, thione, alkyl, a carbocycle or a heterocycle, or two R₃ groups together form a carbocycle or a heterocycle; wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—;
R₄ is independently H or alkyl;
m is 0 to 10; and
n is 0 to 3.

In another aspect of the invention, there are provided compositions comprising compounds of formula I and a carrier, diluent or excipient.

In another aspect of the invention, there is provided a method for inhibiting the signalling of Aurora kinases in a cell comprising contacting said Aurora protein with a compound of formula I.

In another aspect of the invention, there is provided a method for treating a disease or condition in a mammal associated with the signalling of Aurora kinasaes, comprising administering to said mammal an effective amount of a compound of formula I.

### DETAILED DESCRIPTION OF THE PREFERRED EMBODIMENTS

“Alkyl” means a branched or unbranched, saturated or unsaturated (i.e. alkenyl, alkynyl) aliphatic hydrocarbon group, having up to 12 carbon atoms unless otherwise specified. When used as part of another term, for example “alkylamino”, the alkyl portion may be a saturated hydrocarbon chain, however also includes unsaturated hydrocarbon carbon chains such as “alkenylamino” and “alkynylamino. Examples of particular alkyl groups are methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl” “C₁-C₄ alkyl” and “alkyl of 1 to 4 carbon atoms” are synonymous and used interchangeably to mean methyl, ethyl, 1-propyl, isopropyl, cyclopropyl, 1-butyl, sec-butyl or t-butyl. Unless specified, substituted, alkyl groups may contain one, for example two, three or four substituents which may be the same or different. Examples of substituents are, unless otherwise defined, halogen, amino, hydroxyl, protected hydroxyl, mercapto, carboxy, alkoxy, nitro, cyano, amidino, guanidino, urea, sulfonyl, sulfinyl, aminosulfonyl, alkylsulfonylamino, arylsulfonylamino, aminocarbonyl, acylamino, alkoxy, acyl, acyloxy, a carbocycle, a heterocycle. Examples of the above substituted alkyl groups include, but are not limited to; cyanomethyl, nitromethyl, hydroxymethyl, trityloxymethyl, propionyloxymethyl, aminomethyl, carboxymethyl, carboxyethyl, carboxypropyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), 2-carbamoyloxyethyl and the like. The alkyl group may also be substituted with a carbocycle group. Examples include cyclopropylmethyl, cyclobutylmethyl, cyclopentylmethyl, and cyclohexylmethyl groups, as well as the corresponding -ethyl, -propyl, -butyl, -pentyl, -hexyl groups, etc. Substituted alkyls include substituted methyls e.g. a methyl group substituted by the same substituents as the “substituted Cₙ-Cₘ alkyl” group. Examples of the substituted methyl group include groups such as hydroxymethyl, protected hydroxymethyl (e.g. tetrahydropyranyloxymethyl), acetoxymethyl, carbamoyloxymethyl, trifluoromethyl, chloromethyl, carboxymethyl, bromomethyl and iodomethyl.

“Amidine” means the group —C(NH)—NHR wherein R is H or alkyl or aralkyl. A particular amidine is the group —NH—C(NH)—NH₂.

“Amino” means primary (i.e. —NH₂), secondary (i.e. —NRH) and tertiary (i.e. —NRR) amines. Particular secondary and tertiary amines are alkylamine, dialkylamine, arylamine, diarylamine, aralkylamine and diaralkylamine wherein the alkyl is as herein defined and optionally substituted. Particular secondary and tertiary amines are methylamine, ethylamine, propylamine, isopropylamine, phenylamine, benzylamine dimethylamine, diethylamine, dipropylamine and disopropylamine.

“Amino-protecting group” as used herein refers to a derivative of the groups commonly employed to block or protect an amino group while reactions are carried out on other functional groups on the compound. Examples of such protecting groups include carbamates, amides, alkyl and aryl groups, imines, as well as many N-heteroatom derivatives which can be removed to regenerate the desired amine group. Particular amino protecting groups are Boc, Fmoc and Cbz. Further examples of these groups are found in T. W. Greene and P. G. M. Wuts, “Protective Groups in Organic Synthesis”, 2^{nd} ed., John Wiley & Sons, Inc., New York, N.Y., 1991, chapter 7; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected amino” refers to an amino group substituted with one of the above amino-protecting groups.

“Aryl” when used alone or as part of another term means a carbocyclic aromatic group whether or not fused having the number of carbon atoms designated or if no number is designated, up to 14 carbon atoms. Particular aryl groups are phenyl, naphthyl, biphenyl, phenanthrenyl, naphthacenyl, and the like (see e.g. Lang's Handbook of Chemistry (Dean, J. A., ed) 13th ed. Table 7-2 [1985]). A particular aryl is phenyl. Substituted phenyl or substituted aryl means a phenyl group or aryl group substituted with one, two, three, four or five, for example 1-2, 1-3 or 1-4 substituents chosen, unless otherwise specified, from halogen (F, Cl, Br, I), hydroxy, protected hydroxy, cyano, nitro, alkyl (for example C₁-C₆ alkyl), alkoxy (for example C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylaminoalkyl, arylsulfonylamino, arylsulonylaminoalkyl, heterocyclylsulfonylamino, heterocyclylsulfonylaminoalkyl, heterocyclyl, aryl, or other groups specified. One or more methyne (CH) and/or methylene (CH₂) groups in these substituents may in turn be substituted with a similar group as those denoted above. Examples of the term “substituted phenyl” includes but is not limited to a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(iso-propyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such 4-carboxyphenyl; a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino))phenyl. Also, the term “substituted phenyl” represents disubstituted phenyl groups where the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like, as well as trisubstituted phenyl groups where the substituents are different, for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Particular substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. Fused aryl rings may also be substituted with any, for example 1, 2 or 3, of the substituents specified herein in the same manner as substituted alkyl groups.

“Carbocyclyl”, “carbocyclylic”, “carbocycle” and “carbocyclo” alone and when used as a moiety in a complex group such as a carbocycloalkyl group, refers to a mono-, bi-, or tricyclic aliphatic ring having 3 to 14 carbon atoms, for example 3 to 7 carbon atoms, which may be saturated or unsaturated, aromatic or non-aromatic. Particular saturated carbocyclic groups are cyclopropyl, cyclobutyl, cyclopentyl and cyclohexyl groups. A particular saturated carbocycle is cyclopropyl. Another particular saturated carbocycle is cyclohexyl. Particular unsaturated carbocycles are aromatic e.g. aryl groups as previously defined, for example phenyl. The terms “substituted carbocyclyl”, “carbocycle” and “carbocyclo” mean these groups substituted by the same substituents as the “substituted alkyl” group.

“Carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′, 4,4′-tetramethoxybenzhydryl, alkyl such as t-butyl or t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. The species of carboxy-protecting group employed is not critical so long as the derivatized carboxylic acid is stable to the condition of subsequent reaction(s) on other positions of the molecule and can be removed at the appropriate point without disrupting the remainder of the molecule. In particular, it is important not to subject a carboxy-protected molecule to strong nucleophilic bases, such as lithium hydroxide or NaOH, or reductive conditions employing highly activated metal hydrides such as LiAlH₄. (Such harsh removal conditions are also to be avoided when removing amino-protecting groups and hydroxy-protecting groups, discussed below.) Particular carboxylic acid protecting groups are the alkyl (e.g. methyl, ethyl, t-butyl), allyl, benzyl and p-nitrobenzyl groups. Similar carboxy-protecting groups used in the cephalosporin, penicillin and peptide arts can also be used to protect a carboxy group substituents. Further examples of these groups are found in T. W. Greene and P. G. M. Wuts, “Protective Groups in Organic Synthesis”, 2^{nd} ed., John Wiley & Sons, Inc., New York, N.Y., 1991, chapter 5; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981, Chapter 5. The term “protected carboxy” refers to a carboxy group substituted with one of the above carboxy-protecting groups.

“Guanidine” means the group —NH—C(NH)—NHR wherein R is H or alkyl or aralkyl. A particular guanidine is the group —NH—C(NH)—NH₂.

“Hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, benzoyl, acetoxy, carbamoyloxy, benzyl, and silylethers (e.g. TBS, TBDPS) groups. Further examples of these groups are found in T. W. Greene and P. G. M. Wuts, “Protective Groups in Organic Synthesis”, 2^{nd} ed., John Wiley & Sons, Inc., New York, N.Y., 1991, chapters 2-3; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected hydroxy” refers to a hydroxy group substituted with one of the above hydroxy-protecting groups.

“Heterocyclic group”, “heterocyclic”, “heterocycle”, “heterocyclyl”, or “heterocyclo” alone and when used as a moiety in a complex group such as a heterocycloalkyl group, are used interchangeably and refer to any mono-, bi-, or tricyclic, saturated or unsaturated, aromatic (heteroaryl) or non-aromatic ring having the number of atoms designated, generally from 5 to about 14 ring atoms, where the ring atoms are carbon and at least one heteroatom (nitrogen, sulfur or oxygen), for example 1 to 4 heteroatoms. Typically, a 5-membered ring has 0 to 2 double bonds and 6- or 7-membered ring has 0 to 3 double bonds and the nitrogen or sulfur heteroatoms may optionally be oxidized (e.g. SO, SO₂), and any nitrogen heteroatom may optionally be quaternized. Particular non-aromatic heterocycles are morpholinyl (morpholino), pyrrolidinyl, oxiranyl, oxetanyl, tetrahydrofuranyl, 2,3-dihydrofuranyl, 2H-pyranyl, tetrahydropyranyl, thiiranyl, thietanyl, tetrahydrothietanyl, aziridinyl, azetidinyl, 1-methyl-2-pyrrolyl, piperazinyl and piperidinyl. A “heterocycloalkyl” group is a heterocycle group as defined above covalently bonded to an alkyl group as defined above. Particular 5-membered heterocycles containing a sulfur or oxygen atom and one to three nitrogen atoms are thiazolyl, in particular thiazol-2-yl and thiazol-2-yl N-oxide, thiadiazolyl, in particular 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Particular 5-membered ring heterocycles containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Particular benzo-fused 5-membered heterocycles are benzoxazol-2-yl, benzthiazol-2-yl and benzimidazol-2-yl. Particular 6-membered heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are a particular group. Substituents for “optionally substituted heterocycles”, and further examples of the 5- and 6-membered ring systems discussed above can be found in W. Druckheimer et al., U.S. Pat. No. 4,278,793. In a particular embodiment, such optionally substituted heterocycle groups are substituted with hydroxyl, alkyl, alkoxy, acyl, halogen, mercapto, oxo (═O), carboxyl, acyl, halo-substituted alkyl, amino, cyano, nitro, amidino or guanidino. It will be understood that by “optionally substituted” is meant that the heterocycle may be substituted with one or more of the same or different substituents specified. Similarly other groups defind herein that are “optionally substituted” may be substituted with one or more of the specified substituents that may be the same or different.

“Heteroaryl” alone and when used as a moiety in a complex group such as a heteroaralkyl group, refers to any mono-, bi-, or tricyclic aromatic ring system having the number of atoms designated where at least one ring is a 5-, 6- or 7-membered ring containing from one to four heteroatoms selected from the group nitrogen, oxygen, and sulfur, and in a particular embodiment at least one heteroatom is nitrogen (Lang's Handbook of Chemistry, supra). Included in the definition are any bicyclic groups where any of the above heteroaryl rings are fused to a benzene ring. Particular heteroaryls incorporate a nitrogen or oxygen heteroatom. The following ring systems are examples of the heteroaryl (whether substituted or unsubstituted) groups denoted by the term “heteroaryl”: thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothiazolyl, benzothiadiazolyl, benzotriazolyl, benzoimidazolyl and indolyl. A particular “heteroaryl” is: 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-11H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. An alternative group of “heteroaryl” includes; 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl. Heteroaryl groups are optionally substituted as described for heterocycles.

“Inhibitor” means a compound which reduces or prevents the phosphorylation of Aurora kinases or which reduces or prevents the signalling of Aurora kinase. Alternatively, “inhibitor” means a compound which arrests cells in the G2 phase of the cell cycle.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly organic non-toxic bases are isopropylamine, diethylamine, ethanolamine, trimethamine, dicyclohexylamine, choline, and caffeine.

The present invention provides novel compounds having the general formula I:

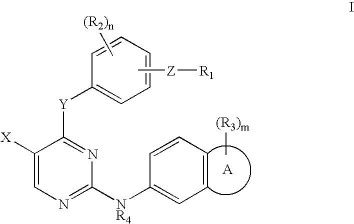

wherein ring A, and X, Y, Z, R₁, R₂, R₃, R₄, m and n are as described herein. It is understood that compounds of the invention encompass salts and solvates thereof. In an embodiment compounds of the invention are hydrates. In an embodiment compounds of the invention are salts.

Ring A is a 5, 6 or 7 member ring carbocycle or heterocycle which is substituted with 0 to 10 R₃ substituents (as valency permits). In a particular embodiment ring A is a 5-7 member carbocycle. In a particular embodiment ring A is a 5-7 member heterocycle. In a particular embodiment the ring A heterocycle contains 1 to 4 heteroatoms selected from N, O, S, SO and SO₂. In an embodiment ring A is substituted with 0 to 5 R₃ substituents. In an embodiment ring A is substituted with 1 to 3 R₃ substituents. In a particular embodiment ring A is a nitrogen containing 5-member ring. In a particular embodiment ring A is a pyrrolidine, oxazolidine, dioxolane, dioxane, imidazolidine, pyrrazole, thiazole, thiazolidine, isothiazole or isothiazolidine ring.

In a particular embodiment ring A is selected from the group consisting of:

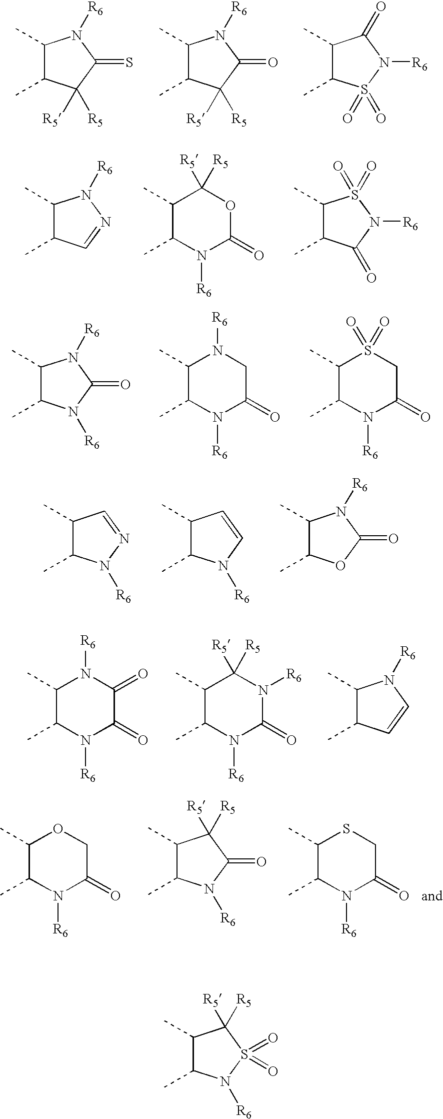

wherein R₅, R_{5′} and R₆ are as defined herein. The dashed lines represent bonds from the benzene ring to which ring A is fused.

In a particular embodiment ring A is selected from the group consisting of:

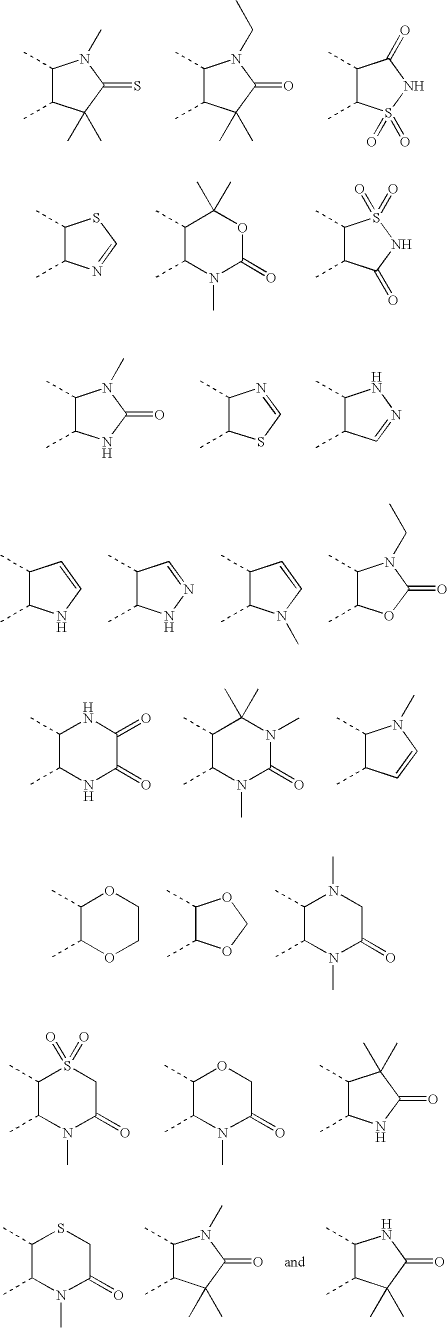

X is H, hydroxyl, halo, amino, nitro, alkyl or haloalkyl. In an embodiment X H. In another embodiment X is haloalkyl, e.g. CF₃. In an embodiment X is OH. In an embodiment X is Cl. In an embodiment X is F.

Y is O, S or NR₄ wherein R₄ is as defined herein. In an embodiment Y is S. In an embodiment Y is O. In an embodiment Y is NR₄ wherein R₄ is H. In an embodiment Y is NR₄ wherein R₄ is alkyl. In a particular embodiment Y is NR₄ wherein R₄ is methyl.

Z is —NR₄C(O)— or —C(O)NR₄—. In a particular embodiment Z is —NR₄C(O)—. In a particular embodiment Z is —C(O)NR₄—. In a particular embodiment Z is located at the para position of the benzene ring to which it is attached. In a particular embodiment Z is at the ortho position of the benzene ring to which it is attached.

R₁ is alkyl, a carbocycle or a heterocycle optionally substituted with hydroxyl, halogen, oxo (═O), amino, carboxyl and alkoxy. In a particular embodiment R₁ is alkyl, cycloalkyl, aryl and heteroaryl each optionally substituted with hydroxyl, halogen, amino, carboxyl or alkoxy. In a particular embodiment, R₁ is cyclopropyl. In a particular embodiment R₁ is alkyl, for example tertiary butyl. In a particular embodiment R₁ is phenyl optionally substituted with halogen. In a particular embodiment R₁ is pyridyl.

R₂ is hydroxyl, halogen, amino, carboxyl or R₂ is alkyl, acyl, alkoxy or alkylthio optionally substituted with hydroxyl, halogen, oxo, thione (═S), amino, carboxyl or alkoxy. In a particular embodiment R₂ is alkyl, alkoxy, hydroxyalkyl, alkylthio, alkoxycarbonyl or aminocarbonyl. In a particular embodiment, R₂ is halogen. In a particular embodiment R₂ is chloro. In a particular embodiment R₂ is CF₃. In a particular embodiment R₂ is alkyl. In a particular embodiment R₂ is methyl.

R₃ is hydroxyl, halogen, amino, oxo, thione, alkyl, a carbocycle or a heterocycle, or two R₃ groups together form a carbocycle or a heterocycle; wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—. It will be understood that a CH₂ group may be replaced at any position along an alkyl chain including a terminal CH₂ group in which case the replacing group is attached to the preceding carbon atom and a following hydrogen. By way of example, CH₂ groups in a propyl substituent may be replaced with —O— in the following different ways: —O—CH₂—CH₃, —CH₂—O—CH₃ or CH₂—CH₂—O—H. It is also understood that “an alkyl group” refers to any alkyl group in the definition of R₃. In a particular embodiment R₃ is alkyl, oxo or thione wherein said alkyl is optionally substituted with halogen, hydroxyl, amino, a carbocycle or a heterocycle and wherein one or more CH₂ groups of an alkyl group is optionally replaced with —O—, —S—, —S(O)—, S(O)₂, —N(R₅)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—. In a particular embodiment R₃ is alkyl wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—. For example, R₃ is alkyl optionally substituted with oxo, thione, amino, hydroxyl, carboxyl or aminocarbonyl. In a particular embodiment R₃ is oxo. In a particular embodiment R₃ is thione. In a particular embodiment R₃ is methyl. In a particular embodiment R₃ is ethyl. In a particular embodiment R₃ is allyl. In a particular embodiment R₃ is isopropyl. In a particular embodiment R₃ is propyl. In a particular embodiment R₃ is ethyloxycarbonylmethyl. In a particular embodiment R₃ is carboxymethyl. In a particular embodiment R₃ is H. In another particular embodiment two R₃ groups together form a carbocycle or a heterocycle. In another particular embodiment two R₃ groups form a spiro carbocycle or heterocycle.

R₄ is in each instance independently H, alkyl, a carbocycle or a heterocycle wherein one or more CH₂ or CH groups of said alkyl is optionally replaced with —O—, —S—, —S(O)—, S(O)₂, —NH—, or —C(O)—; and said alkyl, carbocycle and heterocycle is optionally substituted with hydroxyl, alkoxy, acyl, halogen, mercapto, oxo, carboxyl, acyl, halo-substituted alkyl, amino, cyano nitro, amidino, guanidino an optionally substituted carbocycle or an optionally substituted heterocycle. In a particular embodiment R₄ is H or alkyl. In a particular embodiment R₄ is H. In an embodiment R₄ is alkyl. In an embodiment R₄ is ethyl. In an embodiment R₄ is methyl.

R₅ and R₅′ are independently H, hydroxyl, halogen, amino, oxo, thione, alkyl, a carbocycle or a heterocycle, or R₅ and R_{5′} together form a carbocycle or heterocycle, wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—. It will be understood that a CH₂ group may be replaced at any position along an alkyl chain including a terminal CH₂ group in which case the replacing group is attached to the preceding carbon atom and a following hydrogen. By way of example, CH₂ groups in a propyl substitutent may be replaced with —O— in the following different ways: —O—CH₂—CH₃, —CH₂—O—CH₃ or —CH₂—CH₂—O—H. It is also understood that “an alkyl group” refers to any alkyl group in the definition of R₅. In a particular embodiment R₅ and R_{5′} are independently H, or an optionally substituted alkyl, carbocycle or heterocycle wherein the substituents are halogen, hydroxyl, amino and mercapto and wherein one or more CH₂ groups of said alkyl group is optionally replaced with —O—, —S—, —S(O)—, S(O)₂, —N(R₄)—, —C(O)—, —C(S)—, —C(O)—NR₄—, —NR₄—C(O)—, —SO₂—NR₄—, —NR₄—SO₂—, —NR₄—C(O)—NR₄—, —C(O)—O— or —O—C(O)—. In a particular embodiment R₅ and R_{5′} are independently an optionally substituted carbocycle or heterocycle. In a particular embodiment R₅ and R_{5′} are independently an optionally substituted aryl or heteroaryl ring. In a particular embodiment R₅ and R_{5′} are independently H or alkyl wherein one more CH₂ groups of said alkyl moiety is optionally replaced with —O—, —S—, —S(O)—, S(O)₂, —N(R₄)—, —C(O)—, —C(S)—, —C(O)—NR₄—, —NR₄—C(O)—, —SO₂—NR₄—, —NR₄—SO₂—, —NR₄—C(O)—NR₄—, —C(O)—O— or —O—C(O)—. In a particular embodiment R₅ and R_{5′} are independently alkyl such as methyl. In a particular embodiment R₅ and R_{5′} are both H. In a particular embodiment R₅ and R_{5′} are both methyl. In a particular embodiment R₅ and R_{5′} are both ethyl. In a particular embodiment R₅ and R_{5′} together form a carbocycle (i.e. spiro with respect to the carbon atom from which both R₅ and R_{5′} depend). In a particular embodiment R₅ and R_{5′} together form a cyclopentane ring.

R₆ is alkyl, a carbocycle or a heterocycle, wherein said alkyl, carbocycle and heterocycle are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—. In a particular embodiment R₄ is alkyl optionally substituted with halogen, hydroxyl, amino, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—. It will be understood that a CH₂ group may be replaced at any position along an alkyl chain including a terminal CH₂ group in which case the replacing group is attached to the preceding carbon atom and a following hydrogen. In a particular embodiment R₆ is alkyl wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—. For example, R₆ is alkyl optionally substituted with oxo, thione, amino, hydroxyl, carboxyl or aminocarbonyl. In a particular embodiment R₆ is methyl. In a particular embodiment R₆ is ethyl. In a particular embodiment R₆ is allyl. In a particular embodiment R₆ is isopropyl. In a particular embodiment R₆ is propyl. In a particular embodiment R₆ is ethyloxycarbonylmethyl. In a particular embodiment R₆ is carboxymethyl. In a particular embodiment R₆ is H.

m is 0 to 10. In an embodiment m is 0 to 5. In an embodiment m is 1 to 5. In an embodiment m is 2 to 5. In an embodiment m is 3 to 5.

n is 0 to 5. In an embodiment n is 0 to 3. In an embodiment n is 0 to 2. In an embodiment n is 0 to 1. In a particular embodiment n is 1. In a particular embodiment n is 0.

In an embodiment, compounds of the invention have the general formula IIa:

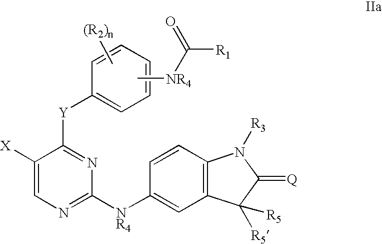

wherein X, Y, Z, R₁, R₂, R₃, R₄, R₅, R_{5′}, and n are as described herein and Q is H₂, O, S or NR₆ wherein R₆ is as described herein. In a particular embodiment Q is O. In another embodiment Q is S. In a particular embodiment Q is H₂ (i.e. two hydrogen atoms pending from the adjacent carbon atom). In a particular embodiment Q is NR₆ in which R₆ is defined herein. In a particular embodiment Q is NR₆ and R₆ is H. In another embodiment Q is NR₆ and R₆ is alkyl. In a particular embodiment Q is NR₆ and R₆ is methyl. In another embodiment —NR₄C(O)—R₁, moiety is at the para position of the benzene ring to which it is attached. In another embodiment —NR₄C(O)—R₁, moiety is at the ortho position of the benzene ring to which it is attached.

In an embodiment, compounds of the invention have the general formula IIb:

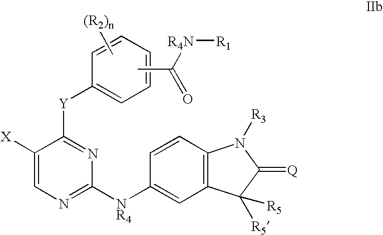

wherein X, Y, Z, R₁, R₂, R₃, R₄, R₅, R_{5′} and n are as described herein and Q is H₂, O, S or NR₆ wherein R₆ is as described herein. In a particular embodiment Q is O. In another embodiment Q is S. In a particular embodiment Q is H₂ (i.e. two hydrogen atoms pending from the adjacent carbon atom). In a particular embodiment Q is NR₆ in which R₆ is defined herein. In a particular embodiment Q is NR₆ and R₆ is H. In another embodiment Q is NR₆ and R₆ is alkyl. In a particular embodiment Q is NR₆ and R₆ is methyl. In another embodiment C(O)NR₄—R₁, moiety is at the para position of the benzene ring to which it is attached. In another embodiment —C(O)NR₄—R₁, moiety is at the ortho position of the benzene ring to which it is attached.

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

The invention also encompasses prodrugs of the compounds described herein. Suitable prodrugs where applicable include known amino-protecting and carboxy-protecting groups which are released, for example hydrolyzed, to yield the parent compound under physiologic conditions. A particular class of prodrugs are compounds in which a nitrogen atom in an amino, amidino, aminoalkyleneamino, iminoalkyleneamino or guanidino group is substituted with a hydroxy (OH) group, an alkylcarbonyl (—CO—R) group, an alkoxycarbonyl (—CO—OR), an acyloxyalkyl-alkoxycarbonyl (—CO—O—R—O—CO—R) group where R is a monovalent or divalent group and as defined above or a group having the formula —C(O)—O—CP1P2-haloalkyl, where P1 and P2 are the same or different and are H, lower alkyl, lower alkoxy, cyano, halo lower alkyl or aryl. In a particular embodiment, the nitrogen atom is one of the nitrogen atoms of the amidino group of the compounds of the invention. These prodrug compounds are prepared reacting the compounds of the invention described above with an activated acyl compound to bond a nitrogen atom in the compound of the invention to the carbonyl of the activated acyl compound. Suitable activated carbonyl compounds contain a good leaving group bonded to the carbonyl carbon and include acyl halides, acyl amines, acyl pyridinium salts, acyl alkoxides, in particular acyl phenoxides such as p-nitrophenoxy acyl, dinitrophenoxy acyl, fluorophenoxy acyl, and difluorophenoxy acyl. The reactions are generally exothermic and are carried out in inert solvents at reduced temperatures such as −78 to about 50 C. The reactions are usually also carried out in the presence of an inorganic base such as potassium carbonate or sodium bicarbonate, or an organic base such as an amine, including pyridine, triethylamine, etc.

Particular compounds of formula I include the following:

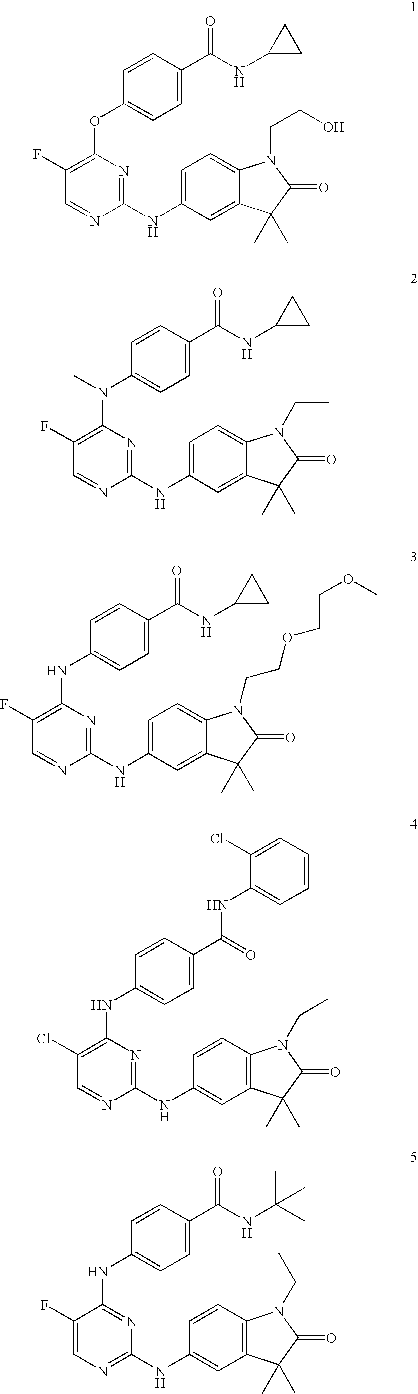

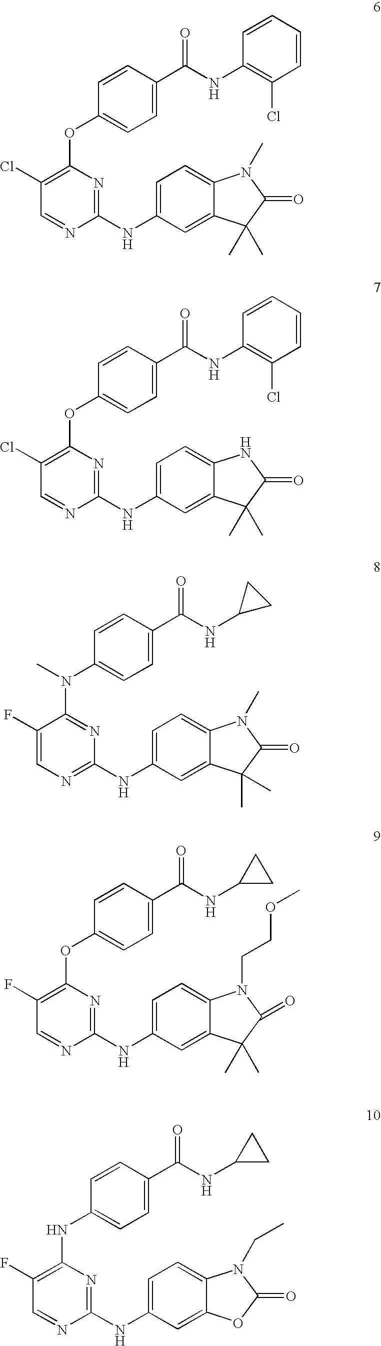

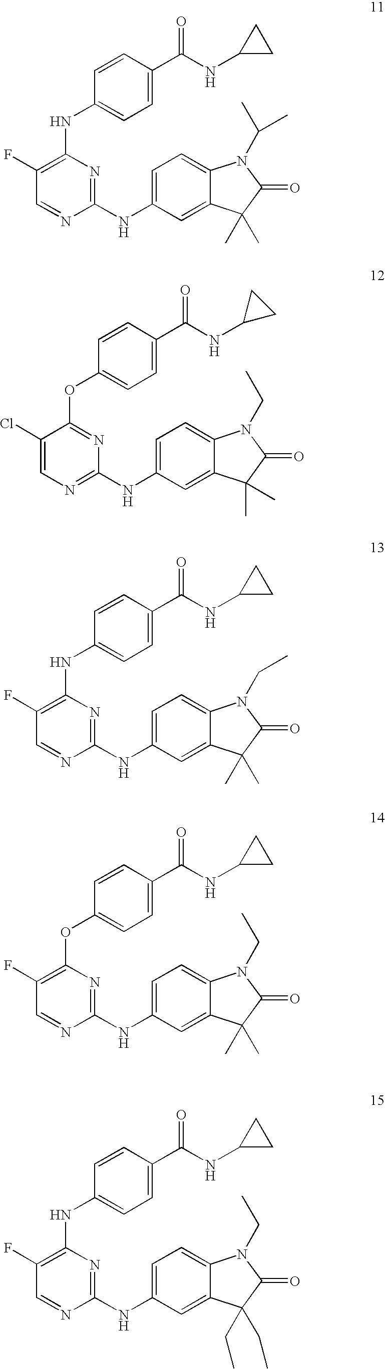

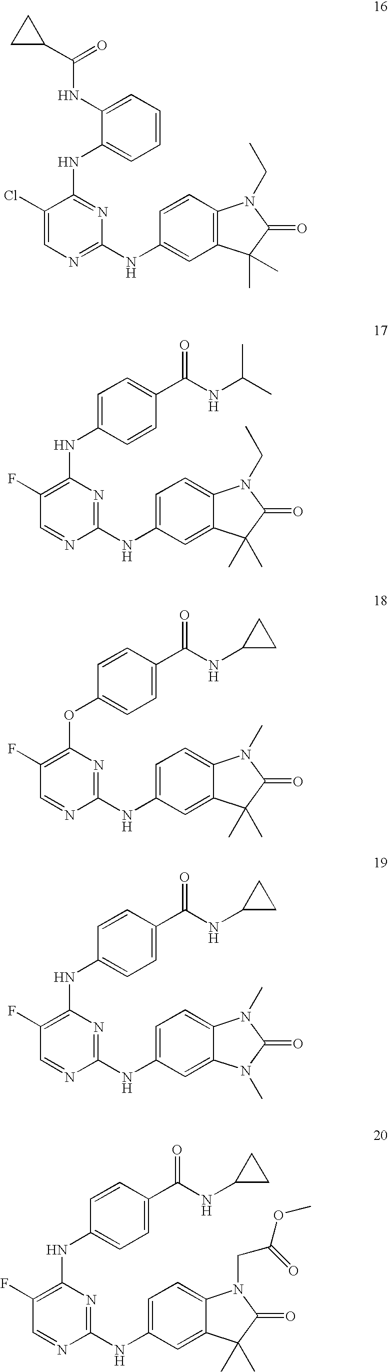

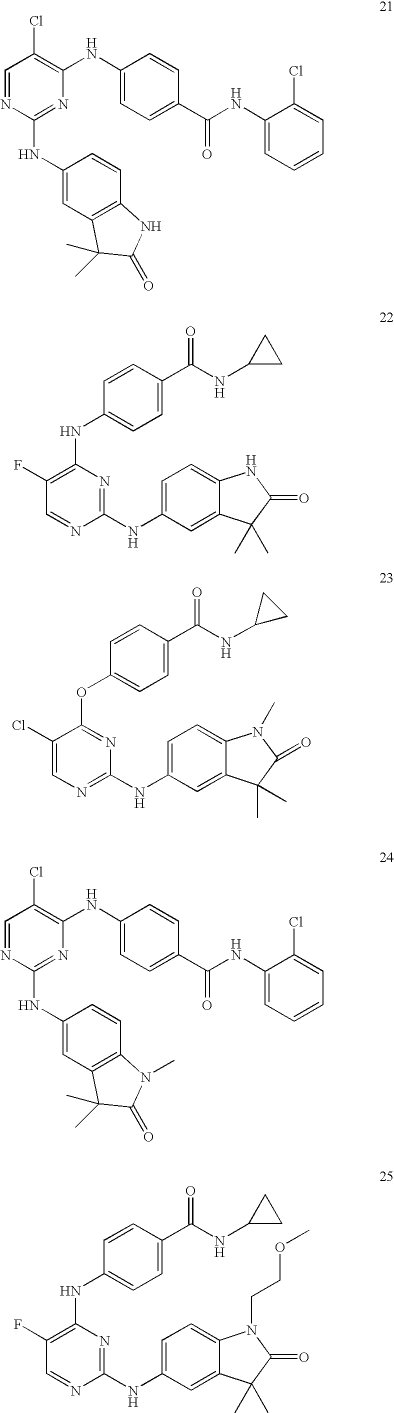

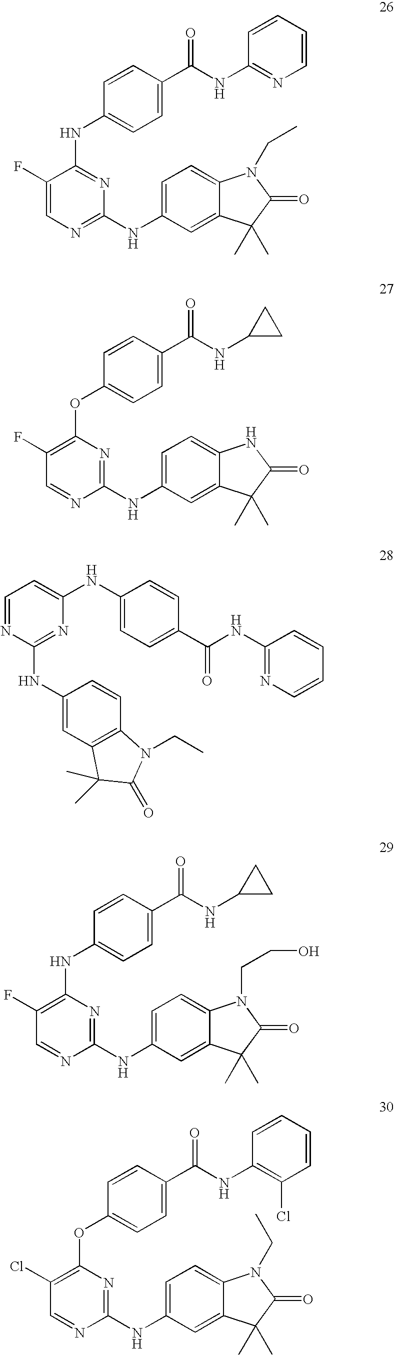

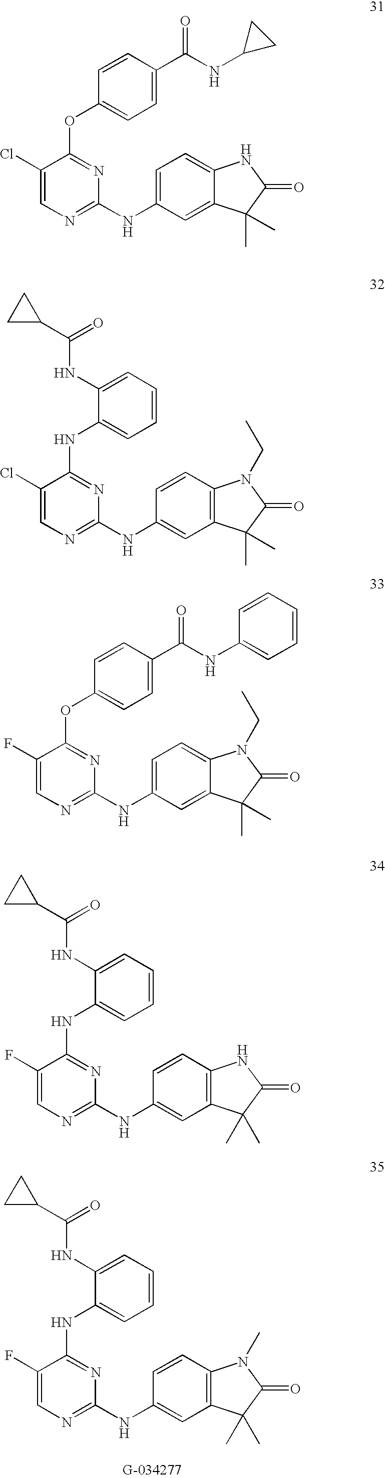

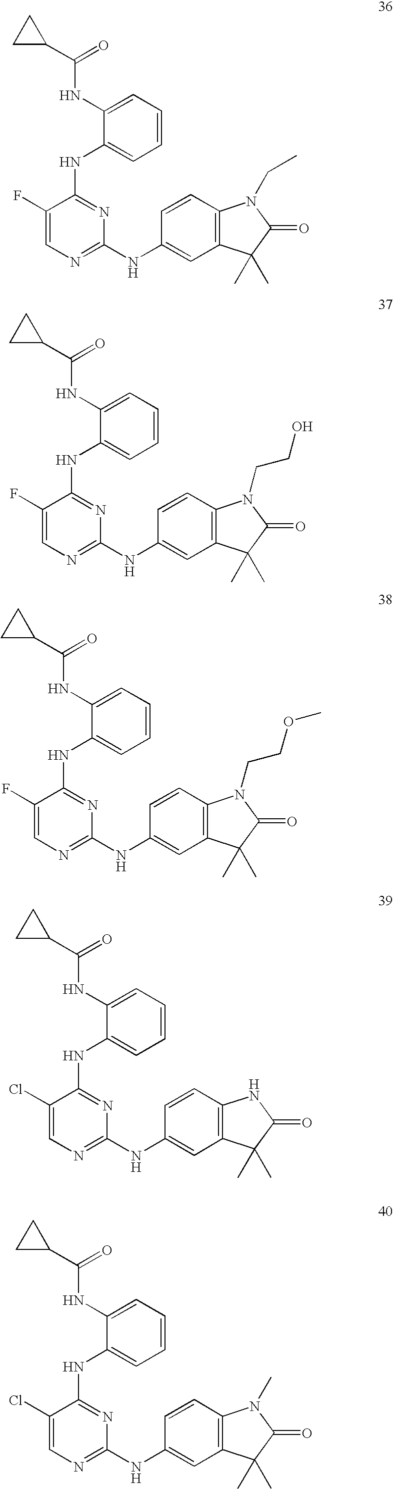

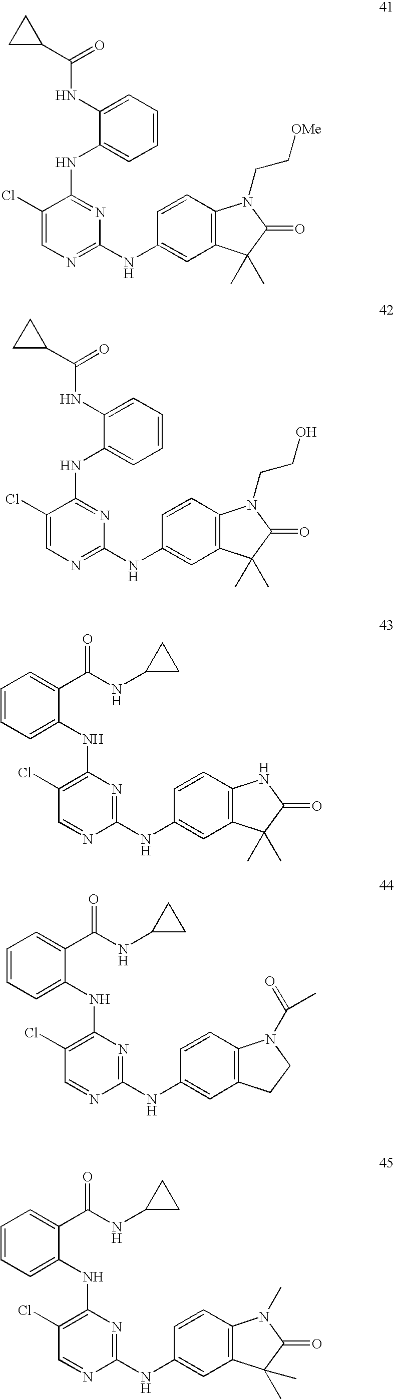

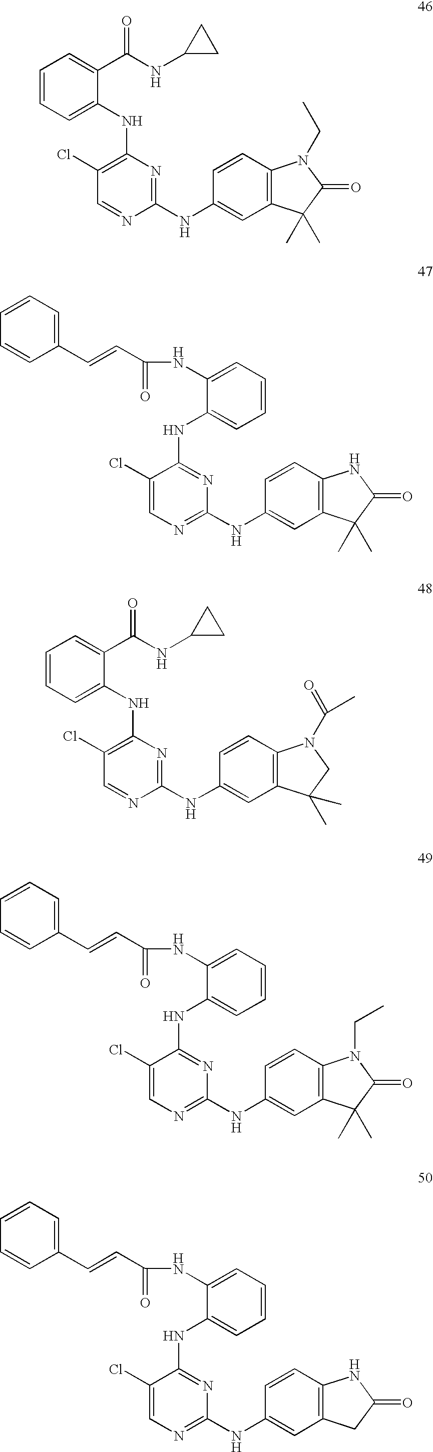

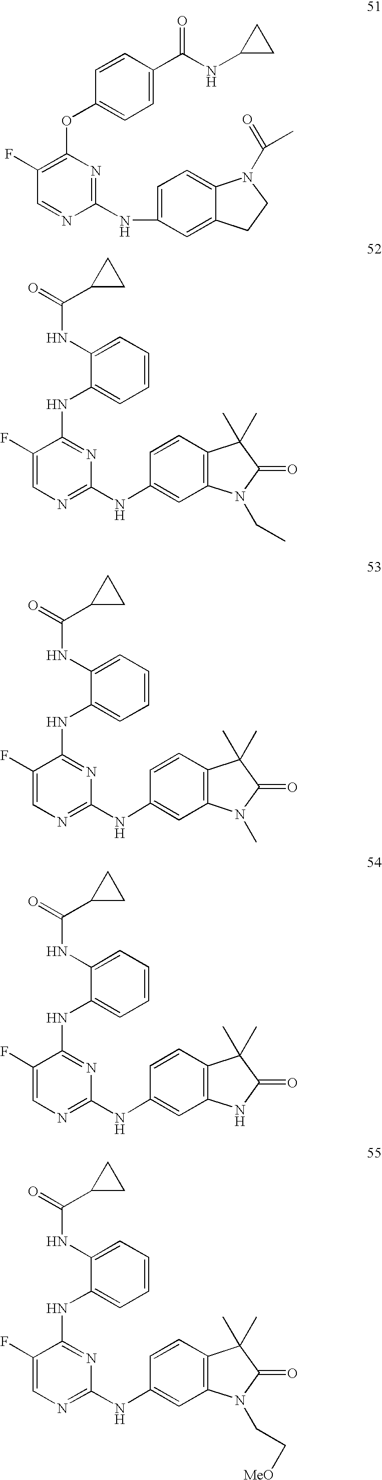

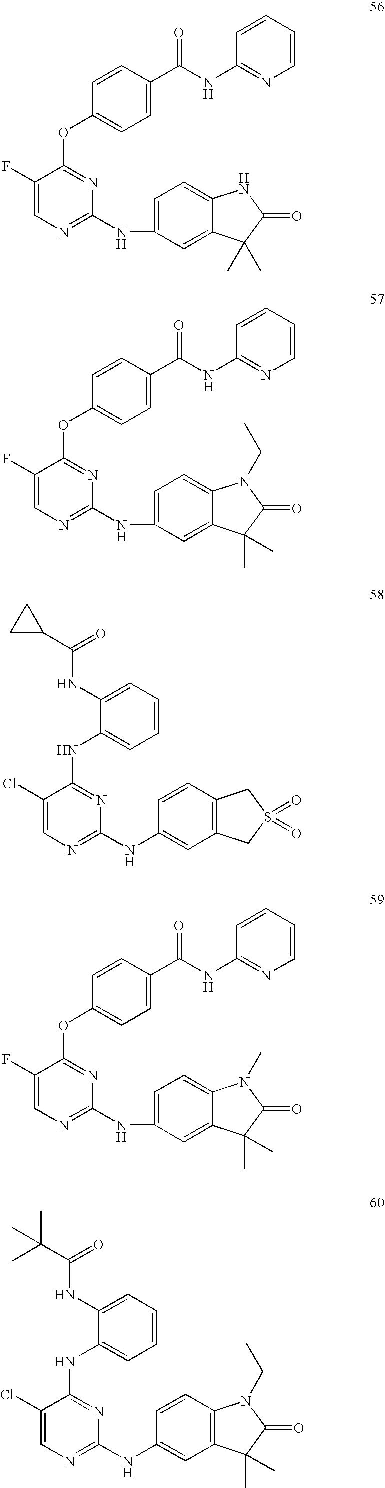

### Synthesis

Compounds of the invention are prepared using standard organic synthetic techniques from commercially available starting materials and reagents. It will be appreciated that synthetic procedures employed will depend on the particular substituents present and that various protection and deprotection steps that are standard in organic synthesis may be required but may not be illustrated in the following general schemes. Compounds of the invention in which Y is NH may be prepared according to the general synthetic scheme 1 in which ring A, X, Z, R₁, R₂, R₃, R₄, m and n are as defined herein.

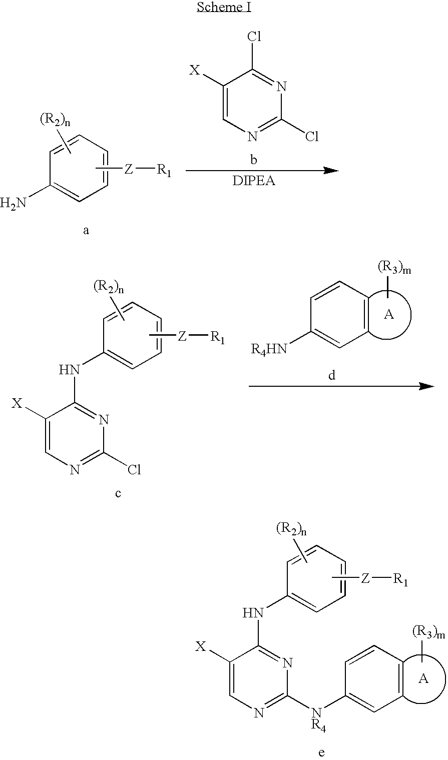

In scheme 1, aniline a is reacted with a 2,4-dichloropyrimidine b to give chloro intermediate c. Chloro intermediate c is then coupled to amine e to give the final product d.

Compounds of the invention in which Y is O may be prepared according to the general synthetic scheme 2 in which ring A, X, Z, R₁, R₂, R₃, R₄, m and n are as defined herein.

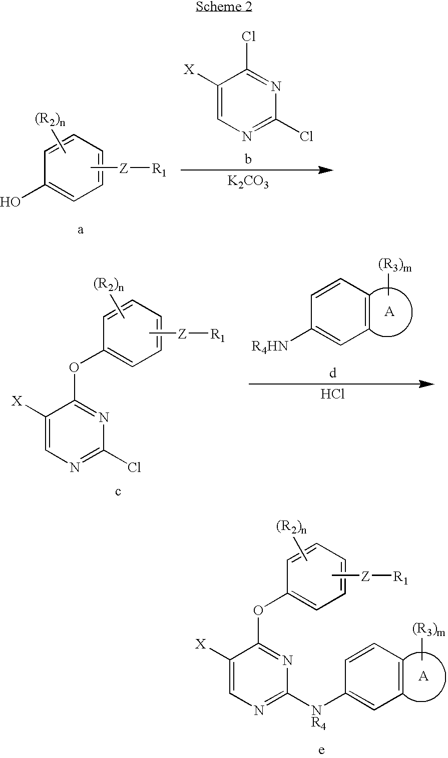

In scheme 2, phenoxy a is reacted with a 2,4-dichloropyrimidine b to give chloro intermediate c. Chloro intermediate c is then coupled to amine d to give the final product e.

In an embodiment, compounds of formula IIb in which Y is NH, may be prepared according to the general scheme 3.

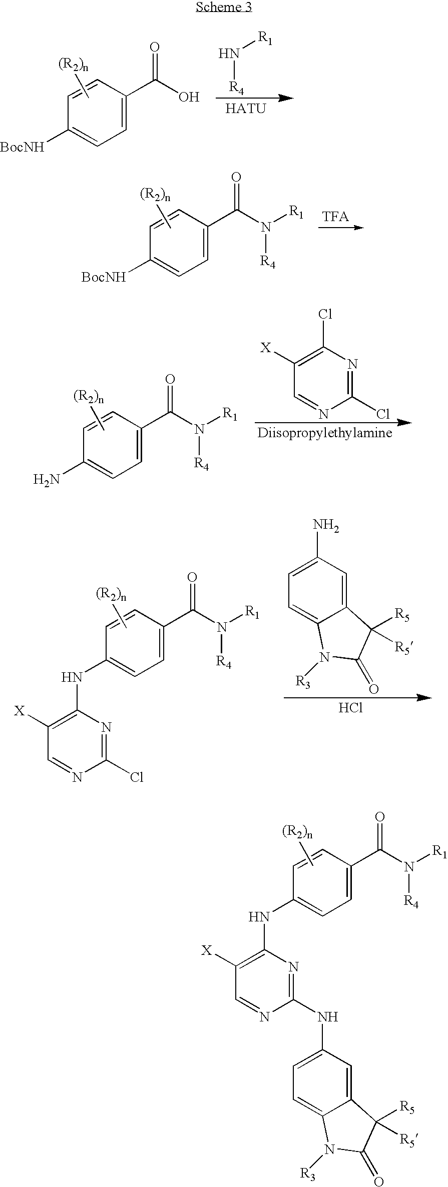

In an embodiment, compounds of formula IIb in which Y is O, may be prepared according to the general scheme 3.

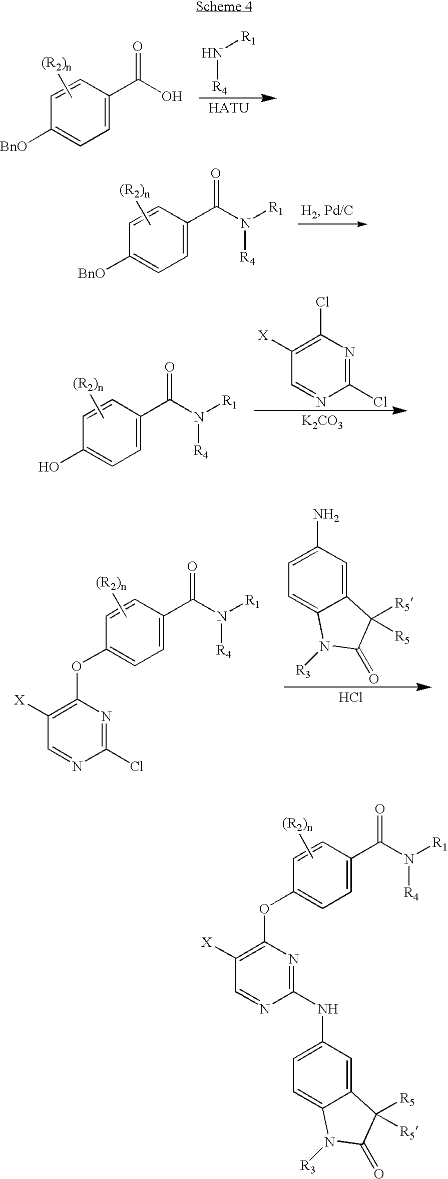

Compounds of formula IIa and IIb incorporate a bicyclic moiety (e.g. a substituted indole or indolone) which may be prepared by coupling a corresponding amine-substituted bicycle with the appropriate chloro-substituted pyrimidine moiety. The amine-substituted bicycle may be prepared from commercially available starting compounds employing standard organic synthetic reactions such as those in the following scheme 5.

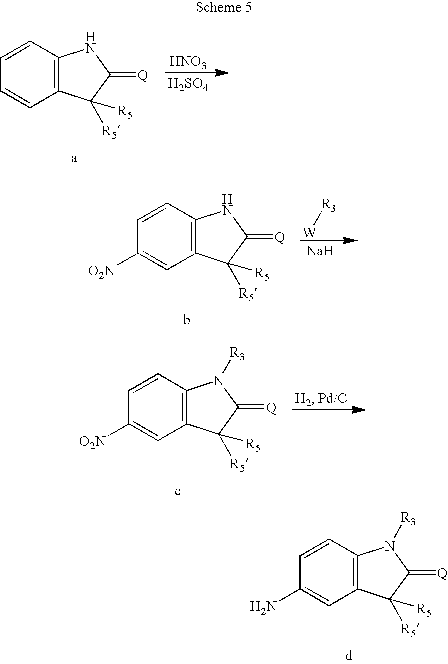

In scheme 5, Q, R₃, R₅, and R₅ are as defined herein and W is a halogen such as I, Br or Cl. Starting compound a is nitrated by reacting with nitric acid and sulfuric acid to give b. The R₃ substituent is introduce by reacting b with halo-substituted R₃ (e.g. R₃—I) and NaH to give c which is subsequently reduced, for example with palladium catalyst to give amine d.

### Indications

The compounds of the invention inhibit Aurora kinase signalling, in particular the phosphorylation of Aurora kinases. Accordingly, the compounds of the invention are useful for inhibiting all diseases associated with the abherant signalling, overexpression and/or amplification of Aurora kinases. Alternatively, compounds of the invention are useful for arresting cells in the G2 phase of the cell cycle. More specifically, the compounds can be used for the treatment of cancers associated with abherant signalling, amplification and/or overexpression of Aurora kinases. Examples of such cancer types include neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma. In particular, compounds of the invention are useful ofr treating colorectal, ovarian, gastric, breast (such as invasive duct adenocarcinomas thereof), renal, cervical, melanoma, lymphoma, bladder, pancreatic, prostate, lung, CNS (such as neuroblastoma), cervical and leukemic cancers.

The compounds may be administered prior to, concomitantly with, or following administration of radiation therapy or cytostatic or antineoplastic chemotherapy. Suitable cytostatic chemotherapy compounds include, but are not limited to (i) antimetabolites, such as cytarabine, fludarabine, 5-fluoro-2′-deoxyuiridine, gemcitabine, hydroxyurea or methotrexate; (ii) DNA-fragmenting agents, such as bleomycin, (iii) DNA-crosslinking agents, such as chlorambucil, cisplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphtheria toxin; (Vi) topoisomerase I poisons, such as camptothecin or topotecan; (vii) topoisomerase II poisons, such as etoposide (VP-16) or teniposide; (viii) microtubule-directed agents, such as colcemid, colchicine, paclitaxel, vinblastine or vincristine; (ix) kinase inhibitors such as flavopiridol, staurosporin, ST1571 (CPG 57148B) or UCN-01 (7-hydroxystaurosporine); (x) miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof, (xi) hormones such as glucocorticoids or fenretinide; (xii) hormone antagonists, such as tamoxifen, finasteride or LHRH antagonists. In a particular embodiment, compounds of the present invention are coadministered with a cytostatic compound selected from the group consisting of cisplatin, doxorubicin, taxol, taxotere and mitomycin C. In a particular embodiment, the cytostatic compound is doxorubicin.

Compounds of the invention may be coadministered with other compounds that induce apoptosis such as ligands to death receptors (“death receptor agonists”). Such agonists of death receptors include death receptor ligands such as tumor necrosis factor a (TNF-α), tumor necrosis factor β (TNF-β, lymphotoxin-α), LT-β (lymphotoxin-β), TRAIL (Apo2L, DR4 ligand), CD95 (Fas, APO-1) ligand, TRAMP (DR3, Apo-3) ligand, DR6 ligand as well as fragments and derivatives of any of said ligands. In an embodiment, the death receptor ligand is TNF-α. In a particular embodiment, the death receptor ligand is Apo2L/TRAIL. Furthermore, death receptors agonists comprise agonistic antibodies to death receptors such as anti-CD95 antibody, anti-TRAIL-R1 (DR4) antibody, anti-TRAIL-R2 (DR5) antibody, anti-TRAIL-R3 antibody, anti-TRAIL-R4 antibody, anti-DR6 antibody, anti-TNF-R1 antibody and anti-TRAMP (DR3) antibody as well as fragments and derivatives of any of said antibodies.

The compounds of the present invention can be also used in combination with radiation therapy. The phrase “radiation therapy” refers to the use of electromagnetic or particulate radiation in the treatment of neoplasia. Radiation therapy is based on the principle that high-dose radiation delivered to a target area will result in the death of reproducing cells in both tumor and normal tissues. The radiation dosage regimen is generally defined in terms of radiation absorbed dose (rad), time and fractionation, and must be carefully defined by the oncologist. The amount of radiation a patient receives will depend on various consideration but the two most important considerations are the location of the tumor in relation to other critical structures or organs of the body, and the extent to which the tumor has spread. Examples of radiotherapeutic agents are provided in, but not limited to, radiation therapy and is known in the art (Hellman, Principles of Radiation Therapy, Cancer, in Principles I and Practice of Oncology, 24875 (Devita et al., 4th ed., vol 1, 1993). Recent advances in radiation therapy include three-dimensional conformal external beam radiation, intensity modulated radiation therapy (IMRT), stereotactic radiosurgery and brachytherapy (interstitial radiation therapy), the latter placing the source of radiation directly into the tumor as implanted “seeds”. These newer treatment modalities deliver greater doses of radiation to the tumor, which accounts for their increased effectiveness when compared to standard external beam radiation therapy.

Ionizing radiation with beta-emitting radionuclides is considered the most useful for radiotherapeutic applications because of the moderate linear energy transfer (LET) of the ionizing particle (electron) and its intermediate range (typically several millimeters in tissue). Gamma rays deliver dosage at lower levels over much greater distances. Alpha particles represent the other extreme, they deliver very high LET dosage, but have an extremely limited range and must, therefore, be in intimate contact with the cells of the tissue to be treated. In addition, alpha emitters are generally heavy metals, which limits the possible chemistry and presents undue hazards from leakage of radionuclide from the area to be treated. Depending on the tumor to be treated all kinds of emitters are conceivable within the scope of the present invention.

Furthermore, the present invention encompasses types of non-ionizing radiation like e.g. ultraviolet (UV) radiation, high energy visible light, microwave radiation (hyperthermia therapy), infrared (IR) radiation and lasers. In a particular embodiment of the present invention UV radiation is applied.

The invention also provides pharmaceutical compositions or medicaments containing the compounds of the invention and a therapeutically inert carrier, diluent or excipient, as well as methods of using the compounds of the invention to prepare such compositions and medicaments. Typically, the compounds of formula I used in the methods of the invention are formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but may range anywhere from about 3 to about 8. Formulation in an acetate buffer at pH 5 is a suitable embodiment. In an embodiment, the inhibitory compound for use herein is sterile. The compound ordinarily will be stored as a solid composition, although lyophilized formulations or aqueous solutions are acceptable.

The composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit Aurora kinase signalling. Such amount may be below the amount that is toxic to normal cells, or the mammal as a whole. Alternatively, “effective amount” of a compound of the invention may be the amount necessary to inhibit the proliferation of cancer cells or the amount required to inhibit the growth of tumours. Generally, the initial pharmaceutically effective amount of the compound of the invention administered parenterally per dose will be in the range of about 0.01-1000 mg/kg, for example about 0.1 to 100 mg/kg of patient body weight per day, with the typical initial range of compound used being 0.3 to 50 mg/kg/day. Oral unit dosage forms, such as tablets and capsules, may contain from about 0.5 to about 1000 mg of the compound of the invention.

The compound of the invention may be administered by any suitable means, including oral, topical, transdermal, parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution is typically filtered, e.g. using a 0.2 micron filter, to remove impurities and contaminants.

### EXAMPLES

The invention will be more fully understood by reference to the following examples. They should not, however, be construed as limiting the scope of the invention. Reagents and solvents were obtained from commercial sources and used as received. ISCO chromatography refers to use of a pre-packed silica gel columns on a Companion system by Teledyne-Isco, Inc. Lincoln, Nebr. The identity and purity of all compounds were checked by LCMS and ¹H NMR analysis.

Abbreviations used herein are as follows:

ACN: acetonitrile;
Chg: cyclohexylglycine;
DCM: dichloromethane
DIPEA: diisopropylethylamine;
DMAP: 4-dimethylaminopyridine;
DME: 1,2-dimethoxyethane;
DMF: dimethylformamide;
DMSO: dimethylsulfoxide
EDC: 1-ethyl-3-(3-dimethylaminopropyl)carbodiimide;
EEDQ: 2-ethoxy-1-ethoxycarbonyl-1,2-dihydroquinoline
LCMS: liquid chromatography mass spectrometry;
LHMDS: lithium hexamethyldisylazide;
HATU: O-(7-Azobenzotriazol-1-yl)-1,1,3,3-tetramethyluronium hexafluorophosphate;

### HOBt: N-Hydroxybenzotriazole

HBTU: 2-(1H-Benzotriazol-1-yl)-1,1,3,3-Tetramethyl-uronium Hexafluorophosphate
HPLC: high performance liquid chromatography;

### NBS: N-bromosuccinamide;

TASF: tris(dimethylamino)sulfonium difluorotrimethylsilicate;
TEA: triethylamine;
TFA: trifluoroacetic acid;
THF: tetrahydrofuran;

### Example 1 5-amino-1-ethyl-3,3-dimethylindolin-2-one

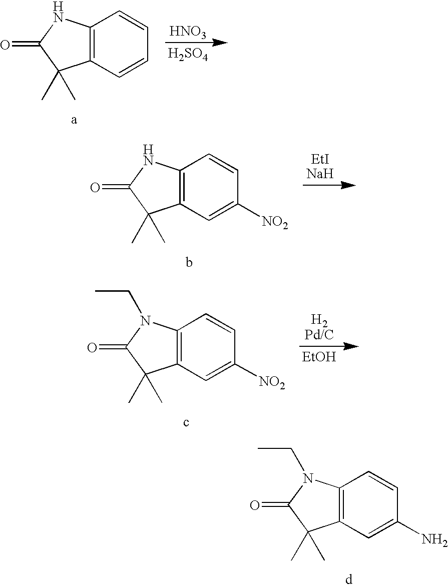

Compound a (38.4 g), prepared according to the procedures described in Robertson et. al. (J. Med. Chem. 29(10) 1832-1840 (1986)), was dissolved in 300 ml of conc. sulfuric acid using mechanical stirring and cooled using a −40° C. cooling bath until stirring became difficult. A solution of 10.1 ml of fuming nitric acid and 50 ml of conc. sulfuric acid was added dropwise and the reaction allowed to warm to ambient temperature with stirring for 12 hours. The reaction mixture was poured into ice water and compound b was collected by filtration and dried (yield: 31 g).

Compound b (9.38 g) was dissolved in 100 ml of DMF and added dropwise to a stirred suspension of sodium hydride (2 g) in 25 ml of DMF. When hydrogen evolution ceased, 4 ml of ethyl iodide was added and the reaction mixture stirred until reaction was complete by tlc. The reaction was partitioned between ethyl acetate and water. The organic extract was concentrated and the crude compound c was recrystallized from ether/hexane (yield: 8.52 g).

Compound c (8.25 g) was reduced under 1 atmosphere of hydrogen in a suspension of 1 g of 10% Pd/C catalyst in 100 ml of methanol with stirring for 18 hours. The catalyst was removed by filtration and evaporation of the solvent gave 5.8 g of compound d (5-amino-1-ethyl-3,3-dimethylindolin-2-one).

### Example 2 5-amino-1,3-dimethyl-1H-benzo[d]imidazol-2(3H)-one

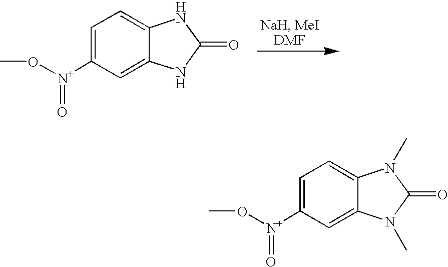

DMF (10 ml) was added to 0.6 g of 60% NaH (15.12 mM) in a 100 mL round bottom flask under N₂. Added to the flask was 1.29 g of 5-nitro-2-benzimidazolinone (7.20 mM) in 10 mL DMF and rinsed with 10 mL more DMF. The solution was stirred 25 minutes and 10.22 g MeI (72 mM) was added and then stirred a further 3 hours. HCl (200 mL, 1 M) was added to the solution and then was extracted with EtOAc, washed with brine, and dried over MgSO₄ and then concentrated in vacuo and flashed 0 to 100% EtOAc in hexanes to give 1.4 g of 1,3-dimethyl-5-nitro-1H-benzo[d]imidazol-2(3H)-one (93% yield).

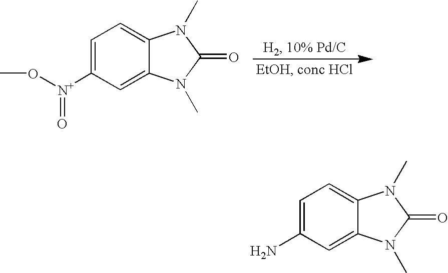

1.4 g of 1,3-dimethyl-5-nitro-1H-benzo[d]imidazol-2(3H)-one was suspended in 100 mL EtOH and 100 uL conc. HCl and 2 scoops of 10% Pd/C was added and an H₂ balloon was attached and stirred overnight. The solution was then filtered through celite and concentrated in vacuo to give 1.27 g of the amine 5-amino-1,3-dimethyl-1H-benzo[d]imidazol-2(3H)-one (106% yield).

### Example 3 methyl 2-(5-amino-3,3-dimethyl-2-oxoindolin-1-yl)acetate

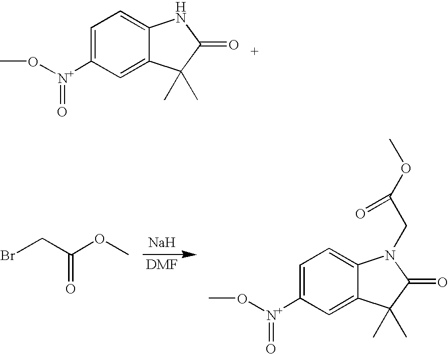

20 mL of DMF was added to 0.87 g of 95% NaH (34.47 mM) in a 250 mL round bottom flask under N₂. 6.46 g of the oxindole (31.34 mM) in 30 mL DMF was then added. The solution was stirred 20 minutes and to it was added 5.27 g methylbromoacetate (34.47 mM) and let stir over night. After concentration in vacuo was added 200 mL 1 M HCl and then extracted with EtOAc, washed with brine, dried over MgSO4, concentrated in vacuo and flashed 0 to 50% with EtOAc in Hexanes to yield 8.14 g of methyl 2-(3,3-dimethyl-5-nitro-2-oxoindolin-1-yl)acetate (93% yield).

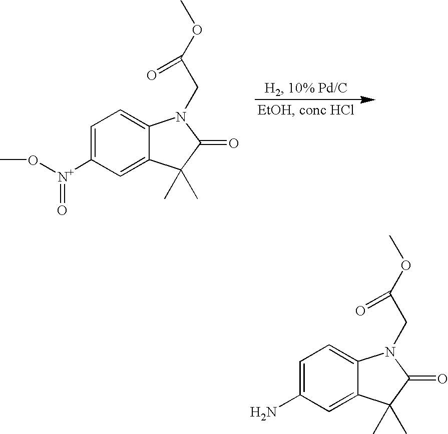

2.3 g of methyl 2-(3,3-dimethyl-5-nitro-2-oxoindolin-1-yl)acetate (8.3 mM) was suspended in 100 mL EtOH, 0.2 mL conc. HCl and approximately 100 mg of 10% Pd/C added, a H₂ balloon was attached and the reaction stirred over night. The solution was then filtered through celite and concentrated in vacuo to give methyl 2-(5-amino-3,3-dimethyl-2-oxoindolin-1-yl)acetate (98% yield).

### Example 4 5-amino-1,3,3-triethylindolin-2-one

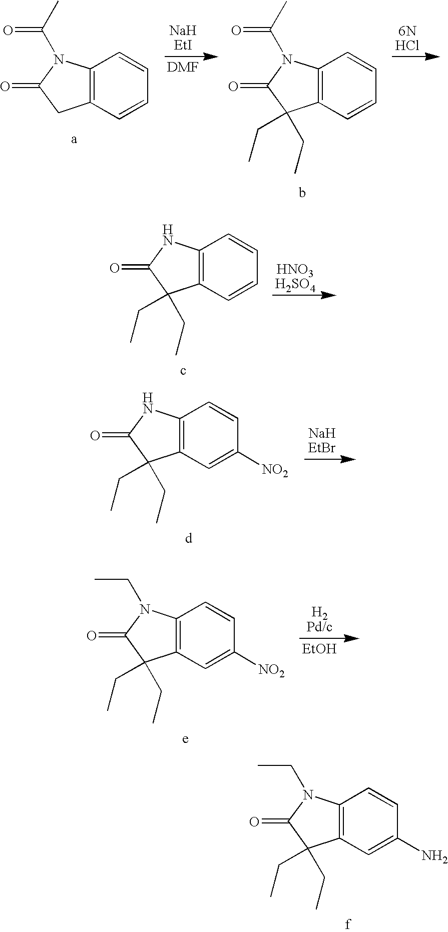

Sodium Hydride (60% dispersion in oil, 4.8 g) was triturated with hexane and decanted twice to remove the oil then suspended in 30 ml of dry DMF with stirring and cooled to 0° C. A solution of 10 g of N-acetyloxindole a in 148 ml of dry DMF was added dropwise over one hour. When hydrogen evolution ceased, 11.4 ml of iodoethane was added over 10 minutes. The reaction mixture was allowed to warm to room temperature and when complete by tlc was poured into ice water and the product extracted with ethyl acetate. The ethyl acetate solution was dried over sodium sulfate, filtered and concentrated and the product purified by automated flash chromatography on silica to give 13.2 g of compound b as a colorless oil.

Compound b (8.43 g) was refluxed in 6N HCl for 2 hrs. at which time a white precipitate had formed and tlc showed the reaction to be complete. The reaction mixture was cooled and the precipitate collected by vacuum filtration, washed with water and dried to give 6.39 g of compound c as a white solid.

Compound c (6.37 g) was suspended in 52 ml of sulfuric acid and cooled until viscous with mechanical stirring on a dry ice/acetonitrile bath. A solution of 1.43 ml of fuming nitric acid in 10.5 ml of sulfuric acid was added over 10 minutes. The reaction was allowed to warm to room temperature. After 6 hrs., the reaction mixture was poured into ice and the precipitated product collected by vacuum filtration. The product was washed with water 2× and vacuum dried to give 7.58 g of compound d.

Compound d (3.46 g) was combined with 9.64 g of cesium carbonate and 1.78 ml of iodoethane in 41 ml of DMF and stirred at 80° C. for 6 hrs. An additional 1.0 ml of iodoethane was added and the reaction mixture maintained at 80° C. with stirring overnight. The reaction was cooled and filtered then partitioned between ethyl acetate and water, washed with brine, dried over magnesium sulfate, filtered and concentrated to give 1.76 g of compound e as a yellow solid.

Compound e (1.2 g) was reduced in one atmosphere of hydrogen (balloon) over 10% Pd/C in methanol (12 ml) for 16 hrs. The catalyst was removed by filtration and the concentrated product recrystallized from hexane and ethyl acetate to give 0.325 g of compound f (5-amino-1,3,3-triethylindolin-2-one) as a tan solid.

### Example 5 5-amino-1-isopropyl-3,3-dimethylindolin-2-one

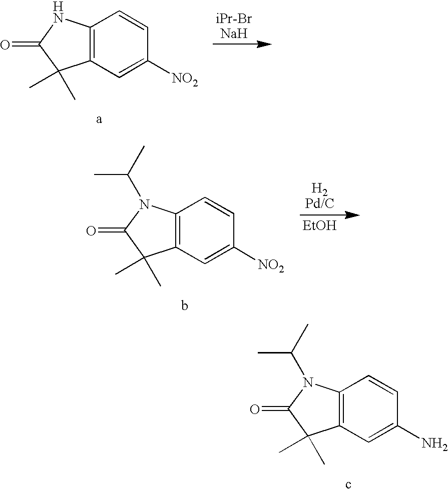

Compound a (1.37 g) was dissolved in 20 ml of DMF and cooled with stirring to 0° C. under nitrogen. Sodium Hydride (239 mg) was added followed 20 min later by 4.1 ml of 2-bromopropane. The reaction mixture was warmed to 60° C. for 4 hrs then concentrated and partitioned between ethyl acetate and 10% citric acid. The organic phase was washed with brine, dried, filtered and concentrated. The crude product was purified by automated flash chromatography on silica to give 1.8 g of compound b.

Compound b (1.8 g) was reduced with hydrogen (balloon) in 50 ml of methanol and 10 ml of acetic acid over 10% palladium on carbon for 2 hrs. The catalyst was removed by filtration through celite. Evaporation of the solvents gave 1.8 g of compound c (5-amino-1-isopropyl-3,3-dimethylindolin-2-one).

### Example 6 5-amino-1-isobutyl-3,3-dimethylindolin-2-one

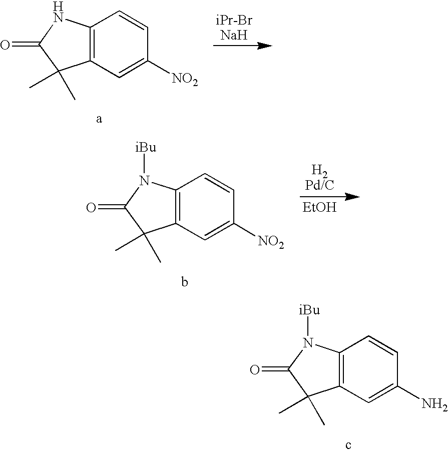

Compound a (1.0 g) was dissolved in 25 ml of dry DMF, cooled to 0°, degassed and blanketed with nitrogen. Sodium hydride (175 mg) was added and the reaction mixture stirred for 30 minutes. Isobultylbromide (3.99 g) was added and the reaction mixture allowed to warm to ambient temperature with stirring overnight. TLC showed reaction complete and the mixture was concentrated and partitioned between 10% citric acid and ethyl acetate. The organic phase was washed with water, brine, dried over sodium sulfate, filtered and concentrated to give 1.19 g of compound b as a brown oil.

Compound b (1.19 g) was reduced under one atmosphere of hydrogen with 10% Pd/C in methanol and 5% acetic acid. The catalyst was filtered off and the solvents evaporated to give 0.99 g of compound c (5-amino-1-isobutyl-3,3-dimethylindolin-2-one) as a brown oil.

### Example 7 1-(2-(2-methoxyethoxy)ethyl)-5-amino-3,3-dimethylindolin-2-one

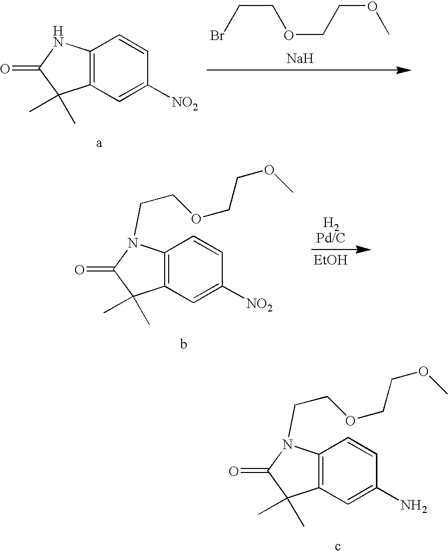

Compound a (1.0 g) was dissolved in 25 ml of dry DMF, degassed and cooled to 0° C. Sodium hydride (175 mg) was added and the reaction stirred for 30 minutes. 1-bromo-2-(2-methoxyethoxy)ethane (3.26 ml) was added and the reaction stirred for 3 hours. The reaction mixture was poured into 10% citric acid and extracted with ethyl acetate. The organic layer was washed with water, brine, dried, filtered and concentrated to give 1.52 g of crude b which was used without purification.

Compound b (1.52 g) was reduced under one atmosphere of hydrogen with 10% Pd/C in 10/1 methanol/acetic acid for 2.5 hours. The catalyst was removed by filtration and the filtrate concentrated to give 1.72 g of compound c (1-(2-(2-methoxyethoxy)ethyl)-5-amino-3,3-dimethylindolin-2-one).

### Example 8 6-amino-3-ethylbenzo[d]oxazol-2(3H)-one

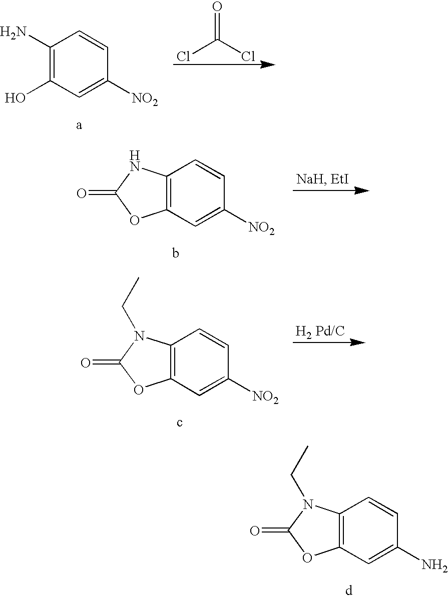

Carbamate b was prepared following the procedures described in John H. Musser, et. al. (J. Med. Chem. 1985, 28, 1255-1259).

Carbamate b (1.29 g, 7.17 mmol) was dissolved in DMF (18 ml) and added dropwise to a cold suspension of sodium hydride (0.60 g) in DMF (18 ml). When hydrogen evolution ceased, ethyl iodide (1.7 ml, 21.5 mmol) was added and the reaction mixture was allowed to stir overnight at room temperature. The reaction mixture was quenched with H₂O and extracted with EtOAc, washed with brine and dried over Na₂SO₄. The solvent was removed in vacuo, and the residue subjected to flash chromatography (silica gel, 0→50% EtOAc in hexanes, gradient elution) to afford c (661 mg, 44%).

Nitro compound c (661 mg, 3.18 mmol) was reduced under 1 atmosphere of hydrogen in a suspension of 10% Pd/C catalyst (1 g) in methanol (7 ml) with stirring for 18 hours. The catalyst was removed by filtration and evaporation of the solvent gave amine d (6-amino-3-ethylbenzo[d]oxazol-2(3H)-one) (530 mg).

### Example 9 Compound 18

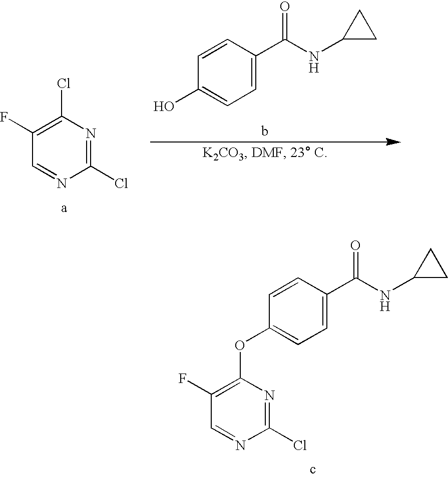

A 100-ml round-bottom flask was charged with pyrimidine chloride a (1.67 g, 10 mmol), followed by phenol b (1.77 g, 10 mmol), anhydrous potassium carbonate (2.76 g, 20 mmol), and DMF (10 ml). The mixture was stirred at 23° C. for 2 hr when the reaction was complete. The mixture was diluted with 100 ml dH₂O to give a white suspension, which was filtered. The white cake was washed with an additional 50 ml dH₂O, azeotroped from 50 ml anhydrous methanol, then 50 ml anhydrous toluene to give pure product c.

c (¹H NMR, CDCl₃) δ (ppm): 8.44 (d, J=2.0 Hz, 1H), 7.93 (d, J=8.8 Hz, 2H), 7.27 (d, J=8.7 Hz, 2H), 2.86 (m, 1H), 0.84 (s, 6H), 0.68 (m, 2H)

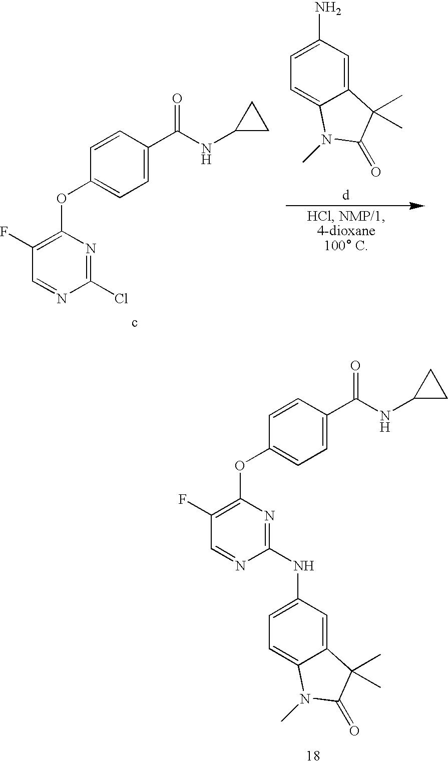

A 10 ml microwave tube with a stirring bar was charged with c (46 mg, 0.15 mmol), followed by d (53 mg, 0.3 mmol, 2.0 equiv.), N-methylpyrrolidinone (0.4 ml), and 1,4-dixoane (2.0 ml). Then a 0.05 ml 4 N solution of HCl in 1,4-dioxane (2.0 equiv.) was added in one portion. The tube was sealed and heated in a 100° C. oil bath for 14 hr when the reaction was complete, as analyzed by LCMS. The mixture was concentrated via rotavap to remove volatile solvent, and the resulting orange thick oil was purified by rpHPLC (0-100% acetonitrile/H₂O) to give compound 18 N-cyclopropyl-4-(5-fluoro-2-(1,3,3-trimethyl-2-oxoindolin-5-ylamino)pyrimidin-4-yloxy)benzamide.

(¹H NMR, CDCl₃) δ (ppm): 8.13 (d, J=3.2 Hz, 1H), 7.89 (d, J=8.7 Hz, 2H), 7.28 (d, J=8.6 Hz, 2H), 7.12 (d, J=8.0 Hz, 1H), 6.61 (d, J=8.0 Hz, 1H), 6.49 (s, 1H), 2.94 (s, 3H), 2.91 (m, 1H), 1.19 (s, 6H), 0.90 (m, 2H), 0.63 (m, 2H).

Compounds 1, 9, 14, 27 and 51 were prepared using analagous procedures using the appropriate amine in the final step. Compounds 56, 57 and 59 were also prepared using an analogous procedure using 4-hydroxy-N-(pyridin-2-yl)benzamide for phenol b and the appropriate amine in the final step.

### Example 10N-(2-(2,5-dichloropyrimidin-4-ylamino)phenyl)-cyclopropanecarboxamide

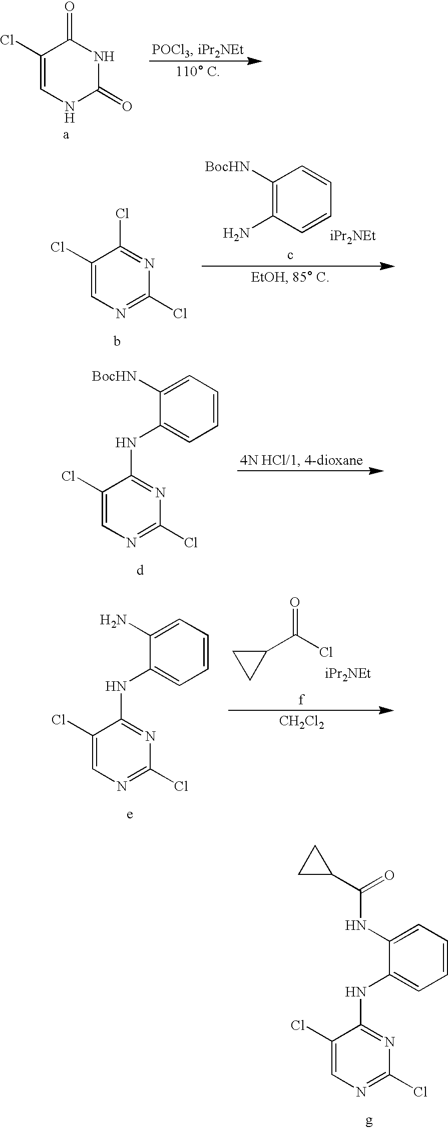

A 500 mL round bottomed flask was charged with 5-chlorouracil a (25.0 g, 170 mmol, 1.0 equiv) and phosphoryl chloride (159 mL, 1.7 mol, 10 equiv). The reaction vessel was equipped with a vigoreaux column followed by careful addition of diisopropylethylamine (59 mL, 340 mmol, 2.0 equiv) over 1 minute. Evolution of white fumes was observed during the addition of diisopropylethylamine. The reaction was then heated to 110° C. and stirred for 3 h. The reaction was cooled to ambient temperature and concentrated in vacuo to crude brown oil. The residual oil was quenched by careful addition of ice chips followed by cold water (100 mL). The aqueous mixture was extracted with diethyl ether and the organic layer washed with brine. The organic layers were dried over anhydrous magnesium sulfate, filtered and concentrated in vacuo to yield crude yellow oil. The crude oil was purified by silica gel chromatography, 0-10% EtOAc/hexane, to provide 2,4,5-trichloropyrimidine b as colorless oil (21.4 g, 69%).

A 1 L round-bottomed flask was charged with 1,2-phenylenediamine (20.0 g, 185 mmol, 1.0 equiv) triethylamine (27.8 mL, 200 mmol, 1.08 equiv) and DMF (372 mL, 0.05 M). To the stirring solution was added 2-tert-butoxycarbonyloxyamino)-2-phenylacetonitrile (49.2 g, 200 mmol, 1.08 equiv). The reaction was then stirred at 55° C. in an oil bath for 12 h when the reaction was deemed complete. The reaction was cooled to ambient temperature and the solution partitioned between toluene (300 mL) and brine (300 mL). The organic layer was extracted with 1.0 N NaOH (aq) (2×250 mL) and brine (250 mL). The organic layer was dried over anhydrous MgSO4, filtered, and concentrated in vacuo to oily brown solid. The crude solid was recrystallized from 1:1 chloroform:hexane to provide tert-butyl 2-aminophenylcarbamate c as off white crystalline solid (13.6 g, 38%)

A 500 mL round bottomed flask was charged with 2,4,5-trichloropyrimidine b (11.9 g, 64.9 mmol, 1.0 equiv), diisopropylethylamine (22.6 mL, 129.8 mmol, 2.0 equiv), and ethanol (238 mL, 0.275 M). To the stirring solution was added tert-butyl 2-aminophenylcarbamate c (13.6 g, 64.9 mmol, 1.0 equiv). The resulting solution was stirred at 85° C. in an oil bath for 12 h when the reaction was deemed complete. The reaction was cooled to ambient temperature and triturated with H₂O (100 mL) causing precipitation of tert-butyl-2-(2,5-dichloropyrmidin-4-ylamino)phenylcarbamate d as white solid. The solid was collected via vacuum filtration then dried to constant weight (21.1 g, 91.5%).

A 250 mL round bottomed flask was charged with compound d (21.1 g, 59.4 mmol, 1.0 equiv) and 4 N HCl in 1,4-dioxane (74 mL, 0.8 M). The resulting homogeneous solution was stirred at ambient temperature for 2 h. The crude reaction was concentrated in vacuo to provide N¹-(2,5-dichloropyrimidin-4-yl)benzene-1,2-diamine e as white solid in HCl salt form (19.8 g, >99%).

A 1 L round bottomed flask was charged with N¹-(2,5-dichloropyrimidin-4-yl)benzene-1,2-diamine e (19.8 g, 60.4 mmol, 1.0 equiv), dichloromethane (431 mL, 0.14 M), and diisopropylethylamine (15.8 mL, 90.5 mmol, 1.5 equiv). To the stirring homogeneous solution was added cyclopropane carbonyl chloride (6.6 mL, 72.4 mmol, 1.20 equiv). The resulting homogeneous solution was stirred at ambient temperature for 12 h until the reaction was deemed complete. The crude solution was concentrated in vacuo to a beige oil. The oil was triturated with methanol (50 mL) and H₂O (150 mL) to yield white precipitated solid. The solid was collected via vacuum filtration and dried under vacuum at 80° C. overnight to provide N-(2-(2,5-dichloropyrimidin-4-ylamino)phenyl)-cyclopropanecarboxamide g (16.8 g, 85.9%).

Compound g was reacted with the appropriate amine using analagous procedure in example 9 give final compounds 16, 32, 39, 40, 41 and 42. Compound 60 was prepared using analogous procedures except using pivaloyl chloride as acid chloride f and the appropriate amine in the final step.

### Example 11 2-(2,5-dichloropyrimidin-4-ylamino)-N-cyclopropylbenzamide

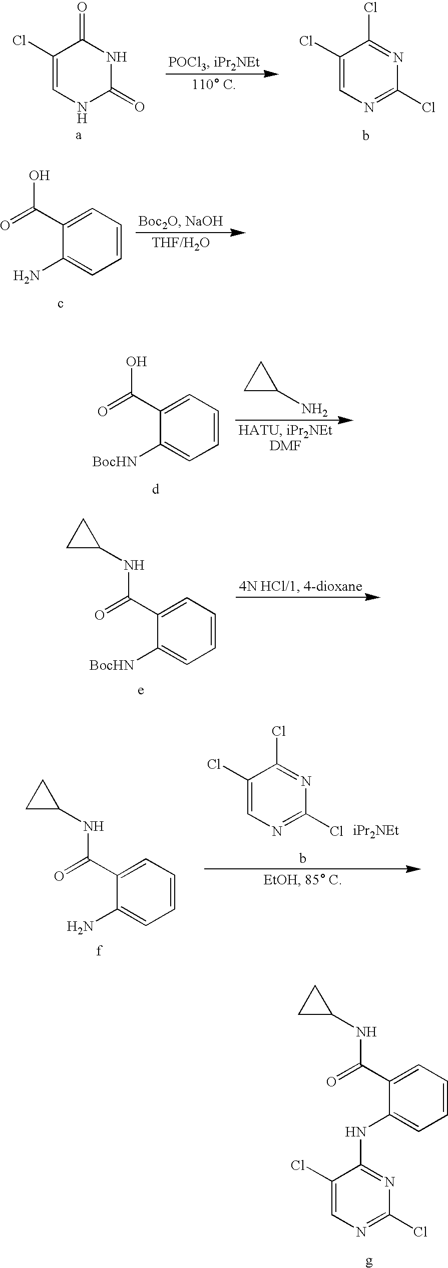

A 500 mL round bottomed flask was charged with 5-chlorouracil a (25.0 g, 170 mmol, 1.0 equiv) and phosphoryl chloride (159 mL, 1.7 mol, 10 equiv). The reaction vessel was equipped with a vigoreaux column followed by careful addition of diisopropylethylamine (59 mL, 340 mmol, 2.0 equiv) over 1 minute. Evolution of white fumes was observed during the addition of diisopropylethylamine. The reaction was then heated to 110° C. and stirred for 3 h. The reaction was cooled to ambient temperature and concentrated in vacuo to crude brown oil. The residual oil was quenched by careful addition of ice chips followed by cold water (100 mL). The aqueous mixture was extracted with diethyl ether and the organic layer washed with brine. The organic layers were dried over anhydrous magnesium sulfate, filtered and concentrated in vacuo to yield crude yellow oil. The crude oil was purified by silica gel chromatography, 0-10% EtOAc/hexane, to provide 2,4,5-trichloropyrimidine b as colorless oil (21.4 g, 69%).

A 1 L round-bottomed flask was charged with 2-aminobenzoic acid c (25.0 g, 182 mmol, 1.0 equiv) and a solution of 1:1 THF:H₂O (364 mL, 0.5 M). The resulting heterogeneous mixture was adjusted to pH 10 by addition of 2N NaOH (aq). Di-tertbutyldicarbonate (43.7 g, 200 mmol, 1.1 equiv) was added to the reaction and the resulting homogeneous solution was stirred at ambient temperature overnight. Following removal of THF, via rotary evaporation, the aqueous solution was adjusted to pH 4 by addition of 15% citric acid, causing precipitation of 2-(tert-butoxycarbonylamino)benzoic acid d as crystalline white solid. The crystalline solid was collected via vacuum filtration and then dried in a vacuum oven (36.0 g, 88%).

A 500 mL round-bottomed flask was charged with 2-(tert-butoxycarbonylamino)benzoic acid d (10.0 g, 42.2 mmol, 1.0 equiv) and 211 mL of DMF (0.2 M). To the resulting homogenous solution was added diisopropylethylamine (8.8 mL, 50.6 mmol, 1.2 equiv) and HATU (17.6 g, 46.4 mmol, 1.1 equiv). The resulting homogeneous solution was stirred at ambient temperature for 5 minutes, followed by addition of cyclopropylamine (5.8 mL, 84.4 mmol, 2.0 equiv). The resulting solution was stirred at ambient temperature for 1 h. The crude reaction was partitioned between ethyl acetate and saturated sodium bicarbonate (2×). The combined organic layers were washed with brine, dried over anhydrous magnesium sulfate, filtered and concentrated in vacuo directly on silica gel. The crude product was purified by silica gel chromatography, 10-50% EtOAc/hexane, to provide tert-butyl 2-(cyclopropylcarbamoyl)phenylcarbamate e as white solid (8.6 g, 74%).

A 100 mL round bottomed flask was charged with tert-butyl 2-(cyclopropylcarbamoyl)-phenylcarbamate e (8.6 g, 31.1 mmol, 1.0 equiv) and 4 N HCl in 1,4-dioxane (50 mL, 0.6 M, 6.5 equiv). The resulting homogeneous solution was stirred at ambient temperature for 2 h. The crude reaction was concentrated in vacuo to provide 2-amino-N-cyclocpropylbenzamide f as white solid in HCl salt form (6.7 g, >99%).

A 500 mL round bottomed flask was charged with 2-amino-N-cyclopropylbenzamide f (6.7 g, 38.4 mmol, 1.0 equiv), diisopropylethylamine (13.4 mL, 76.8 mmol, 2.0 equiv), and ethanol (140 mL, 0.275 M). To the resulting homogeneous suspension was added 2,4,5-trichloropyrimidine b (6.9 g, 38.4 mmol, 1.0 equiv). The resulting solution was stirred at 85° C. in an oil bath overnight. The reaction was cooled to ambient temperature and treated with water (100 mL), causing the precipitation of 2-(2,5-dichloropyrimidin-4-ylamino)-N-cyclopropylbenzamide g as white solid. The white solid was collected via vacuum filtration then dried in a vacuum oven (7.5 g, 61%). Compound g, was reacted with the appropriate amine according to the analagous procedure in example 9 to give compounds 43, 44, 45, 46 and 48. Compound 4, 21 and 24 were prepared according to an analagous procedure except using 4-aminobenzoic acid as compound c and reacting the resulting Boc-protected intermediate with 2-chloroaniline instead of cyclopropylamine and reacting the resulting intermediate corresponding to g with the appropriate amine.

### Example 12 Compound 37

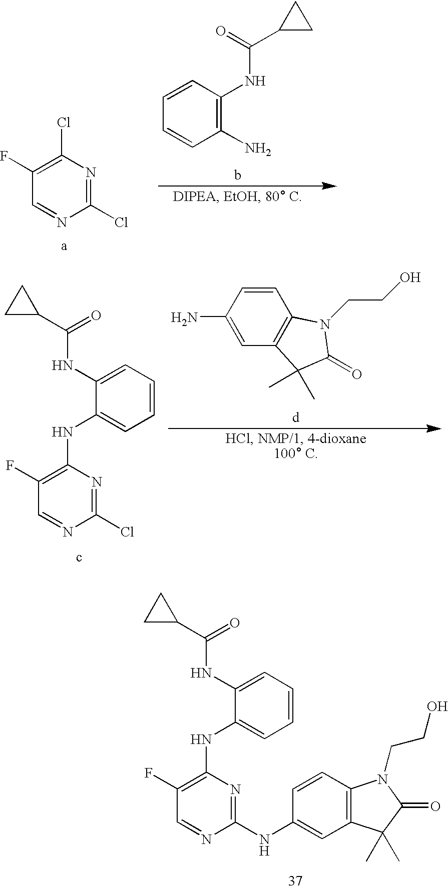

A 50-ml round-bottom flask was charged with 2,4-dichloro-5-fluoropyrimidine a (1.02 g, 6.1 mmol), followed by N-(2-aminophenyl)cyclopropanecarboxamide b (1.07 g, 6.1 mmol), DIPEA (2.12 mL, 12.2 mmol), and anhydrous ethanol (15 ml). The mixture was heated in an oil bath at 80° C. for 7 hr. The reaction mixture was concentrated, and then diluted with 50 ml EtOAc, washed with sat. NH₄Cl (2×25 mL), dried over Na₂SO₄, filtered and concentrated via rotavap. The crude product was purified by flash chromatograph with 0-80% EtOAc/Hexane to give N-(2-(2-chloro-5-fluoropyrimidin-4-ylamino)phenyl)cyclopropane-carboxamide c as a white solid.

A 10-ml microwave tube with a stirring bar was charged with c (62 mg, 0.2 mmol), followed by 5-amino-1-(2-hydroxyethyl)-3,3-dimethylindolin-2-one d (48 mg, 0.22 mmol), 1-butanol (2.0 mL) and conc. HCl (15 uL). The tube was sealed and heated in a 100° C. oil bath until the reaction was complete as analyzed by LCMS. The mixture was concentrated via rotavap to remove volatile solvent, and the resulting thick oil was purified by rpHPLC (0-100% acetonitrile/H₂O) to give N-(2-(5-fluoro-2-(1-(2-hydroxyethyl)-3,3-dimethyl-2-oxoindolin-5-ylamino)pyrimidin-4-ylamino)phenyl)cyclopropanecarboxamide (compound 37).

Compounds 34, 35, 36, 38, 52, 53, 54, 55 were prepared according to procedures analagous to those for preparing compound 37.

### Example 13 Aurora A & Aurora B In Vitro Kinase Assays

Kinase activities were measured by Enzyme-Linked Immunosorbent Assay (ELISA): Maxisorp 384-well plates (Nunc) were coated with recombinant fusion protein comprising residues 1-15 of Histone H3 fused to the N-terminus of Glutathione-S-Transferase. Plates were then blocked with a solution of 1 mg/mL I-block (Tropix Inc) in phosphate-buffered saline. Kinase reactions were carried out in the wells of the ELISA plate by combining an appropriate amount of Aur A and B kinases and/or mutants thereof with test compound and 30 μM ATP. The reaction buffer was 1× Kinase Buffer (Cell Signaling Technologies) supplemented with 1 μg/mL I-block. Reactions were stopped after 45 minutes by addition of 25 mM EDTA. After washing, substrate phosphorylation was detected by addition of anti-phospho-Histone H3 (Ser 10) 6G3 mAb (Cell Signaling cat #9706) and sheep anti-mouse pAb-HRP (Amersham cat# NA931V), followed by calorimetric development with TMB.

### Example 14 Cellular Proliferation/Viability Assay

Potency of test compounds in inhibiting cellular proliferation and/or cellular viability was estimated using a cellular ATP assay (Cell-Titer-Glo, Promega). Cells (HCT116, HT29 colon cancer cell lines, MCF-7 breast cancer cell line) were seeded in 384-well plates (Greiner μClear) at an appropriate density in 50:50 DMEM/Hams F-12 medium supplemented with 10% fetal calf serum, and allowed to attach overnight. Test compounds were sequentially diluted in DMSO and then culture medium, and added to the cells at appropriate concentrations. Cells were incubated with compound for 5 days. Cell number/viability was estimated using Cell-Titer-Glo reagent (Promega) according to manufacturers instructions.

### Example 15 Cellular PhosphoHistone/Mitosis Assay

Efficacy of compounds in inhibiting progression through mitosis and Aurora B-dependent Histone H3 phosphorylation was estimated by automated microscopy and image analysis. HT29 colon cancer cells were seeded at an appropriate density in 384-well plates (Greiner μClear) in 50:50 DMEM/Hams F-12 medium supplemented with 10% fetal calf serum and allowed to attach overnight. Test compounds were sequentially diluted in DMSO and then culture medium, and added to the cells at appropriate concentrations. After 16 hours of incubation with compounds, cells were processed for immunofluorescent microscopy. Cells were fixed with 4% paraformaldehyde, then wells are blocked with 5% fish gelatin (Sigma), then incubated with anti-phospho-Histone H3 (Ser 10) rabbit polyclonal antibody (Cell Signaling) and anti-MPM2 monoclonal antibody (Cell Signaling), followed by incubation with goat anti-rabbit-AlexaFluor 555 and sheep anti-mouse AlexaFluor 488 (Invitrogen) and nuclear counterstaining with Hoechst 33342. Images were acquired using a Discovery-1 automated microscopy system (Molecular Devices), and analyzed using MetaMorph software (Molecular Devices) to calculate the percentage of cells scoring positive for MPM2 and for Phospho-Histone H3.

Compounds of the invention that were tested in the ELISA assay were found to inhibit aurora A and/or aurora B kinase activity with an IC₅₀ of less than 0.5 μM. For example, aurora A kinase activity was inhibit by compound 5 with an IC₅₀ of 0.0108 μM, compound 26 with an IC₅₀ of 0.0231 μM, compound 38 with an IC₅₀ of 0.0072 μM, compound 44 with an IC₅₀ of 0.0021 μM, compound 48 with an IC₅₀ of 0.0248 μM, compound 50 with an IC₅₀ of 0.0060 μM, compound 58 with an IC₅₀ of 0.0006 μM and compound 59 with an IC₅₀ of 0.0104 μM. In a particular embodiment, compounds of the invention inhibit aurora A and or aurora B kinase activity with an with an IC₅₀ of less than 0.2 μM. In a particular embodiment, compounds of the invention inhibit aurora A and or aurora B kinase activity with an with an IC₅₀ of less than 0.1 M. In a particular embodiment, compounds of the invention inhibit aurora A and or aurora B kinase activity with an with an IC₅₀ of less than 0.05 μM. In a particular embodiment, compounds of the invention inhibit aurora A and or aurora B kinase activity with an with an IC₅₀ of less than 0.01 M.

Alternatively, compounds of the invention that were tested in the cellular proliferation/viability assays were found to inhibit HCT116, HT29 and/or MCF-7 cell proliferation and/or viability with an IC₅₀ of less than 25 μM. For example, compound 49 inhibited HCT116 cell proliferation with an IC₅₀ of 0.1104 and HT29 with an IC₅₀ of 0.100. In a particular embodiment, compounds of the invention inhibit HCT116, HT29 and/or MCF-7 cell proliferation and/or viability with an IC₅₀ of less than 1 μM. In a particular embodiment, compounds of the invention inhibit HCT116, HT29 and/or MCF-7 cell proliferation and/or viability with an IC₅₀ of less than 0.5 μM. In a particular embodiment, compounds of the invention inhibit HCT116, HT29 and/or MCF-7 cell proliferation and/or viability with an IC₅₀ of less than 0.1 M. In a particular embodiment, compounds of the invention inhibit HCT116, HT29 and/or MCF-7 cell proliferation and/or viability with an IC₅₀ of less than 0.05 μM.

Alternatively, compounds of the invention that were tested in the phosphohistone assay were found to inhibit progression of HT29 cells through mitosis and aurora B-dependent hostine H3 phosphorylation with an IC₅₀ of less than 10 μM. In an embodiment, compounds of the invention inhibit progression of HT29 cells through mitosis and aurora B-dependent histone phosphorylation with an IC₅₀ of less than 5 μM. In an embodiment, compounds of the invention inhibit progression of HT29 cells through mitosis and aurora B-dependent histone phosphorylation with an IC₅₀ of less than 0.5 μM. In an embodiment, compounds of the invention inhibit progression of HT29 cells through mitosis and aurora B-dependent histone phosphorylation with an IC₅₀ of less than 0.1 μM. In an embodiment, compounds of the invention inhibit progression of HT29 cells through mitosis and aurora B-dependent histone phosphorylation with an IC₅₀ of less than 0.05 μM.

We claim:

1. A compound of formula I: 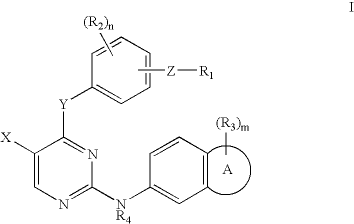
wherein
ring A is a 5, 6 or 7 member ring carbocycle or heterocycle;
X is H, hydroxyl, halo, amino, nitro, alkyl or haloalkyl;
Y is O or NR₄;
Z is —NR₄C(O)— or —C(O)NR₄—;
R₁ is alkyl, a carbocycle or a heterocycle optionally substituted with hydroxyl, halogen, oxo, amino, carboxyl or alkoxy;
R₂ is hydroxyl, halogen, amino, carboxyl or is alkyl, acyl, alkoxy or alkylthio optionally substituted with hydroxyl, halogen, oxo, thione, amino, carboxyl or alkoxy;
R₃ is hydroxyl, halogen, amino, oxo, thione, alkyl, a carbocycle or a heterocycle, or two R₃ groups together form a carbocycle or a heterocycle; wherein said alkyl, carbocycles and heterocycles are optionally substituted with halogen, hydroxyl, carboxyl, amino, alkyl, a carbocycle or a heterocycle; and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—;
R₄ is independently H or alkyl;
m is 0 to 10;
n is 0 to 5;
and salts and solvates thereof.

2. The compound of claim 1, wherein ring A is selected from the group consisting of:

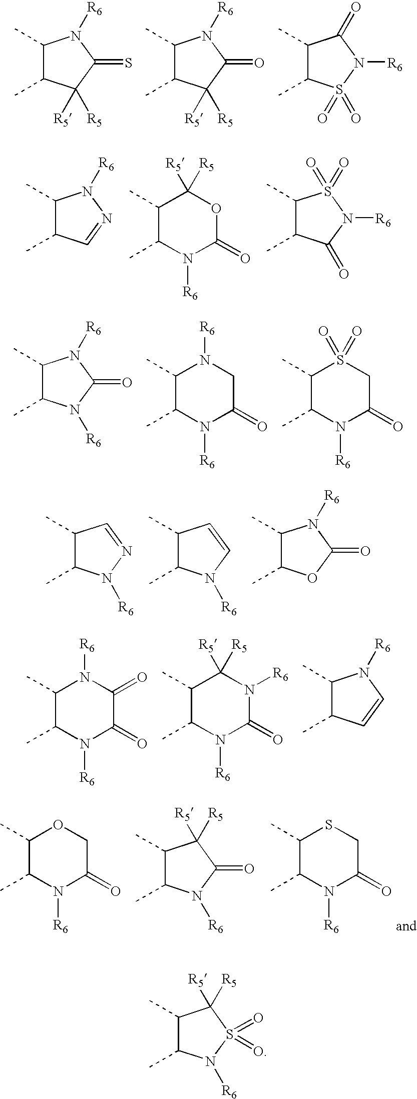

3. The compound of claim 1, wherein ring A is selected from the group consisting of:

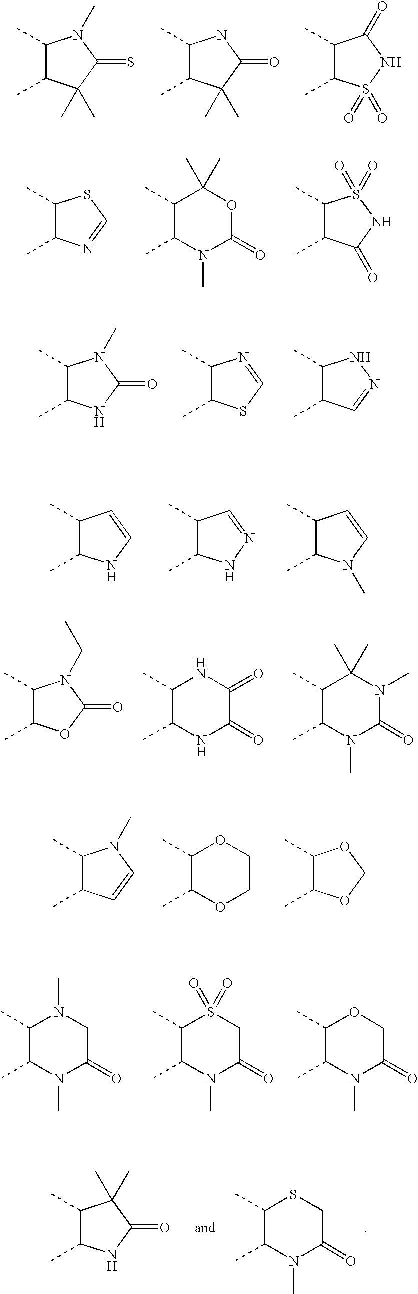

4. The compound of claim 1, wherein R₁ is alkyl, cycloalkyl, aryl and heteroaryl each optionally substituted with hydroxyl, halogen, amino, carboxyl or alkoxy.

5. The compound of claim 1, wherein X is halogen.

6. The compound of claim 1, wherein Y is NH.

7. The compound of claim 1, wherein Y is O.

8. The compound of claim 1, wherein Z is —NHC(O)—.

9. The compound of claim 1, wherein Z is —C(O)NH—.

10. The compound of claim 1, wherein R₃ is alkyl optionally substituted with oxo, thione, amino, hydroxyl, carboxyl or aminocarbonyl.

11. The compound of claim 1, wherein said compound has the general formula IIa 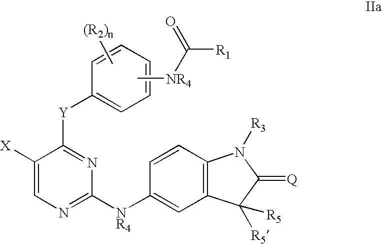
X is H, hydroxyl, halo, amino, alkyl or haloalkyl;
Y is O, S or NR₄;
Q is H₂, O, S or NR₆;
R₁ is alkyl, a carbocycle or a heterocycle optionally substituted with hydroxyl, halogen, oxo, amino, carboxyl or alkoxy;
R₂ is hydroxyl, halogen, amino, carboxyl or is alkyl, acyl, alkoxy or alkylthio optionally substituted with hydroxyl, halogen, oxo, thione, amino, carboxyl or alkoxy;
R₃ is hydroxyl, halogen, amino, oxo, thione, alkyl, a carbocycle or a heterocycle, or two R₃ groups together form a carbocycle or a heterocycle; wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—;
R₄ is independently H or alkyl;
R₅ and R₅′ are independently H, hydroxyl, halogen, amino, oxo, thione, alkyl, a carbocycle or a heterocycle, or R₅ and R_{5′} together form a carbocycle or heterocycle, wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—.
R₆ is alkyl, a carbocycle or a heterocycle, wherein said alkyl, carbocycle and heterocycle are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—.
m is 0 to 10; and
n is 0 to 5.

12. The compound of claim 11, wherein Q is O.

13. The compound of claim 11, wherein Q is NR₆ and R₆ is alkyl optionally substituted with halogen, hydroxyl, amino, a carbocycle or a heterocycle and wherein one or more CH₂ groups of an alkyl group is optionally replaced with —O—, —S—, —S(O)—, S(O)₂, —N(R₅)—, —C(O)—, —C(O)—NR₅—, —NR₅—C(O)—, —SO₂—NR₅—, —NR₅—SO₂—, —NR₅—C(O)—NR₅—, —C(O)—O— or —O—C(O)—.

14. The compound of claim 1, wherein said compound has the general formula IIb 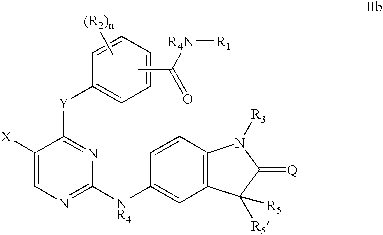
X is H, hydroxyl, halo, amino, alkyl or haloalkyl;
Y is O or NR₄;
Q is H₂, O, S or NR₆;
R₁ is alkyl, a carbocycle or a heterocycle optionally substituted with hydroxyl, halogen, oxo, amino, carboxyl or alkoxy;
R₂ is hydroxyl, halogen, amino, carboxyl or is alkyl, acyl, alkoxy or alkylthio optionally substituted with hydroxyl, halogen, oxo, thione, amino, carboxyl or alkoxy;
R₃ is hydroxyl, halogen, amino, oxo, thione, alkyl, a carbocycle or a heterocycle, or two R₃ groups together form a carbocycle or a heterocycle; wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—;
R₄ is independently H or alkyl;
R₅ and R₅′ are independently H, hydroxyl, halogen, amino, oxo, thione, alkyl, a carbocycle or a heterocycle, or R₅ and R_{5′} together form a carbocycle or heterocycle, wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—.
R₆ is alkyl, a carbocycle or a heterocycle, wherein said alkyl, carbocycle and heterocycle are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₄)—, —C(O)—, —C(O)—NR₄—, —NR₄—C(O)—, —SO₂—NR₄—, —NR₄—SO₂—, —NR₄—C(O)—NR₄—, —C(O)—O— or —O—C(O)—.
m is 0 to 10;
n is 0 to 5;
and salts and solvates thereof.

15. The compound of claim 14, wherein Q is O.

16. The compound of claim 14, wherein Q is NR₆ and R₆ is alkyl optionally substituted with halogen, hydroxyl, amino, a carbocycle or a heterocycle and wherein one or more CH₂ groups of an alkyl group is optionally replaced with —O—, —S—, —S(O)—, S(O)₂, —N(R₅)—, —C(O)—, —C(O)—NR₅—, —NR₅—C(O)—, —SO₂—NR₅—, —NR₅—SO₂—, —NR₅—C(O)—NR₅—, —C(O)—O— or —O—C(O)—.

17. A method of treating cancer in a mammal comprising administering an effective amount of a compound of claim 1.

18. A method of inhibiting the proliferation of a tumor cell comprising contacting said tumor cell with a compound of claim 1.

19. A method for treating a disease or condition in a mammal associated with the Aurora kinase signalling, comprising administering to said mammal an effective amount of a compound of claim 11.

20. A method for treating a disease or condition in a mammal associated with the Aurora kinase signalling, comprising administering to said mammal an effective amount of a compound of claim 14.
